(19) United States
(12) Patent Application Publication
(10) Pub. No.: US 2012/0330258 A1
(43) Pub. Date: Dec. 27, 2012

(54) SHEET MATERIALS HAVING IMPROVED SOFTNESS AND GRAPHICS CLARITY

(76) Inventor: Simon Poruthoor, Alpharetta, GA (US)

(21) Appl. No.: 13/370,655

(22) Filed: Feb. 10, 2012

Related U.S. Application Data
(63) Continuation-in-part of application No. 13/169,633, filed on Jun. 27, 2011.

(51) Int. Cl. A61F 13/15; B31D 1/00; B32B 3/10

(52) U.S. Cl. 604/365; 428/195.1; 493/320

(57) ABSTRACT

Disclosed herein is an article including a fibrous nonwoven layer attached to a film layer to form a laminate with an ink composition applied to a surface of the film layer which is opposite the nonwoven layer to form graphics. In use, such laminates are often positioned so that the fibrous nonwoven layer forms the visible side of the laminate with the ink composition being partially obscured by the film and nonwoven layers of the laminate. At least the surface of the film layer opposite the ink composition is treated with a softening agent which improves the visibility and clarity of the underlying graphics. Processes for forming such articles are also disclosed.

### CLAIM OF BENEFIT OF PRIORITY

The present application is a continuation-in-part and claims benefit of priority to U.S. patent application Ser. No. 13/169,633 filed on Jun. 27, 2011, the contents of which are incorporated herein.

### BACKGROUND

Absorbent articles such as diapers, training pants, feminine hygiene products and incontinence products typically include an outercover constructed from a substrate laminate of a liquid impermeable film and a nonwoven fabric constructed from hydrophobic polymeric fibers. These outercovers may be printed with graphics or other indicia that enhance the aesthetic appeal of the absorbent article. The graphics may be printed in a number of locations relative to the exterior surface of the product and their location can affect the ability of the graphics to be seen. One location for the printing of such graphics is on the exterior surface of the fibrous nonwoven web. Unfortunately, while printing the graphics in this location can increase the visibility of the graphics, it can also affect the durability of the graphics. Durability of the printed graphics is important to reduce the chance that the graphic may rub off of the outercover onto, for example, the skin of the wearer or the caretaker, or any other item the outercover may contact. It also has been found, that the process of printing graphics on the exterior surface of the substrate may negatively impact the soft feel of the substrate.

Accordingly, when the graphics are printed on the exterior surface of the product, there is a need to improve the softness of printed substrates to maintain the aesthetic quality of the overall product. This is especially true in the context of absorbent articles used for personal hygiene application including the products mentioned above. Additionally, there exists a need to improve the softness of printed substrates without negatively impacting the durability of an ink composition applied to the nonwoven surface of a film-web laminate, such as those useful as an outercover of an absorbent article.

In certain applications, it has been found to be more desirable to locate the printing/graphics on an interior surface of the film-web laminate. In so doing, the graphics can be protected by being separated from the exterior-most surface of the product which is the surface which sees the most abrasion during use. As a result, it is possible to locate the graphics on the inner surface of the fibrous web portion of the film-web laminate. Alternatively, the printing/graphics may be located on the interior or exterior surface of the film portion of the film-web laminate.

As the printing/graphics are moved further and further away from the exterior surface of the film-web laminate and thus the exterior surface of the overall product, the ability to visually see the printing/graphics and, if necessary, interpret their meaning becomes more and more difficult. As a result, there is a need to ensure not only that the exterior surface of the film-web laminate remains soft but also that the clarity of the printing/graphics is maintained as much as possible.

When the printing/graphics are printed on the interior side of the film portion of the film-web laminate or even on a subjacent layer, it becomes even more important to ensure that the transparency of the film and other intervening layers is maximized as well as the clarity and vibrancy of the printing/graphics. The present invention is directed to improving both the softness of such film-web laminates and also improving the visibility of the printing/graphics associates with such laminates.

### SUMMARY OF THE INVENTION

Objects and advantages of the invention will be set forth in part in the following description, or may be obvious from the description, or may be learned through practice of the invention.

In one embodiment, an article defining a visible surface includes a sheet material, an ink composition overlaying the sheet material, and a softening agent overlaying the ink composition, wherein the ink composition is positioned between the sheet material and the softening agent. For example, the softening agent may be erucamide, cetyl 2-ethylhexanone, ethylhexyl stearate, ethylhexyl hydroxyetarate, and so forth. The basis weight of the softening agent on the sheet material may be from about 0.1 to about 6 grams per square meter, optionally from about 0.1 to about 4 grams per square meter. The basis weight of the ink composition may be from about 0.01 to about 10 grams per square meter, optionally from about 0.01 to about 2 grams per square meter. The basis weight of the sheet material may be from about 6 to about 50 grams per square meter.

In one aspect, the sheet material may include synthetic polymer fibers interlaid to form a nonwoven web. As one example, the nonwoven web may include polyolefin fibers.

In a further aspect, the ink composition may include a crosslinking agent in an amount greater than about 3.5% by weight based on the dried weight of the ink composition, wherein the crosslinking agent includes an aziridine oligomer with at least two aziridine functional groups. The ink composition may further include an acrylic colloidal dispersion polymer.

In an even further aspect, the nonwoven web may be laminated to a breathable film comprising micropores such that the visible surface of the nonwoven web is opposite of the breathable film.

In another embodiment, an absorbent article may include a liquid permeable topsheet, an absorbent core, and a liquid impermeable backsheet, wherein the absorbent core is positioned between the topsheet and the backsheet. The backsheet may include a nonwoven web overlying a film layer such that the film layer faces the absorbent core and the nonwoven web defines a visible surface that includes synthetic polymer fibers interlaid to form a nonwoven web, an ink composition overlaying the synthetic polymer fibers of the nonwoven web, and, a softening agent overlying the ink composition, wherein the ink composition is positioned between the synthetic polymer fibers and the softening agent. The softening agent may be erucamide, cetyl 2-ethylhexanone, ethylhexyl stearate, ethylhexyl hydroxyetarate, and so forth. The basis weight of the softening agent may be from about 0.1 to about 6 grams per square meter, optionally from about 0.1 to about 4 grams per square meter.

In a further embodiment, a method of providing a soft, printed nonwoven web includes the steps of providing a nonwoven web of synthetic fibers, wherein the nonwoven web has a visible surface, applying an ink composition to the visible surface of the nonwoven web to form a printed surface, and applying a softening agent to the printed surface. The softening agent may be erucamide, cetyl 2-ethylhexanone, ethylhexyl stearate, ethylhexyl hydroxyetarate, and so forth. The softening agent may be, for example, flexographically printed or ink jet printed on the nonwoven web. In some aspects the softening agent may be slot coated on to the nonwoven web, solvent spray coated on to the nonwoven web, thermal spray coated on to the nonwoven web, meltblown on to the nonwoven web, placed in controlled or patterned areas on the nonwoven web, or applied to the nonwoven web as a monolithic film or particulates.

In other aspects, the ink composition may include a crosslinking agent in an amount of greater than about 1.0% by weight, optionally greater than about 3.5% by weight, based on the dried weight of the ink composition, wherein the crosslinking agent comprises an aziridine oligomer with at least two aziridine functional groups, and further wherein the method further comprises the step of crosslinking the ink composition after applying the softening agent.

In other embodiments the ink composition is printed on the underside or inner surface of the film layer to form graphics thereon and the outer surface (the surface adjacent the fibrous nonwoven layer) is treated with a softening agent to improve the transparency of the film and thus the vibrancy and visibility of the graphics on the underside of the film when viewed from the nonwoven side of the film-nonwoven laminate. In this regard, an article is provided which defines a visible surface having a film having an inner surface and an outer surface. A fibrous nonwoven web having an outer visible surface and an inner surface is also provided with the inner surface of the fibrous nonwoven web being attached to the outer surface of the film to form a laminate having a visible surface defined by the visible outer surface of the fibrous nonwoven web. An ink composition is overlaid upon the inner surface of the film to form a graphic thereon and a softening agent is overlaid upon at least a portion of the outer surface of the film. The resultant laminate has a water vapor transmission rate of between about 1,000 and about 20,000 grams per square meter per day. In addition, the laminate has at least one of an opacity between about 40 and about 70 or an optical density ratio between about 30 and about 80.

The ink composition in some instances is only applied to certain areas of the underside of the film layer. As a result, the softening agent if desired may only be applied to those areas of the outer surface of the film which are in general registry with the ink composition so that the ink composition and the softening agent are in vertical juxtaposition with one another. This will in turn provide other areas of the laminate which are devoid of softening agent which from an economic point-of-view may be desirable as transparency of non-printed areas of the laminate is not necessarily critical and may provide a cost savings.

The softening agent can be applied directly to the film layer but it can also be applied to the fibrous nonwoven web which can have the added benefit of improving the softness of the fibrous nonwoven web. When improving the visibility and clarity of the film layer, the concentration of the add-on of the softening agent to the laminate is generally in the range of about 0.5 to about 3.0 grams per square meter of laminate.

A number of softening agents can be used to improve the transparency of the film layer including silicone and silicone-based compounds such as polysiloxane, straight chain silicone, branched chain silicone, functionalized silicone, dimethicone, amino functional siloxane, dimethyl, methylglycol siloxane and methyhydrogen siloxane.

Depending on the attributes of the softening agents available, the film layer may be treated with one softening agent and the fibrous nonwoven layer may be treated with a different softening agent thereby imparting different properties to each of the two layers.

The article defined herein has a wide variety of uses including, but not limited to, absorbent articles which typically include a liquid permeable topsheet, a backsheet and an absorbent core disposed between the topsheet and the backsheet. The backsheet may comprise a film having an inner surface and an outer surface along with a fibrous nonwoven web having an outer visible surface and an inner surface with the inner surface of the fibrous nonwoven web being attached to the outer surface of the film to form a laminate having a visible surface defined by the visible outer surface of the fibrous nonwoven web. An ink composition overlies the inner surface of the film to form a graphic thereon and a softening agent overlies at least a portion of the outer surface of the film. The laminate has a water vapor transmission rate of between about 1,000 and about 20,000 grams per square meter per day and it has at least one of an opacity between about 40 and about 70 or an optical density ratio between about 30 and about 80.

On method for forming such an article includes providing a film having an inner surface and an outer surface and providing a fibrous nonwoven web having an outer visible surface and an inner surface. The inner surface of the fibrous nonwoven web is attached to the outer surface of the film to form a laminate having a visible surface defined by the outer visible surface of the fibrous nonwoven web. An ink composition is applied to the inner surface of the film to form graphics thereon and a softening agent is applied to the outer surface of the film in sufficient quantity such that the laminate has from about 0.5 to about 3.0 grams per square meter of the softening agent, such that the laminate has a water vapor transmission rate of between about 1,000 and about 20,000 grams per square meter per day and such that the laminate has at least one of an opacity between about 40 and about 70 or an optical density ratio of between about 30 and about 80.

Another method for forming such an article includes providing a film having an inner surface and an outer surface and providing a fibrous nonwoven web having an outer visible surface and an inner surface. The inner surface of the fibrous nonwoven web is attached to the outer surface of the film to form a laminate having a visible surface defined by the outer visible surface of the fibrous nonwoven web. An ink composition is applied to the inner surface of the film to form graphics thereon and a softening agent is applied to the fibrous nonwoven web such that at least a portion of the softening agent is present on the outer surface of the film. The softening agent is applied in sufficient quantity such that the laminate has from about 0.5 to about 3.0 grams per square meter of the softening agent, such that the laminate has a water vapor transmission rate of between about 1,000 and about 20,000 grams per square meter per day and such that the laminate has at least one of an opacity between about 40 and about 70 or an optical density ratio of between about 30 and about 80.

In both forms of the method, the softening agent may applied to the outer surface of the film in such a manner that the pattern of application of the softening agent is in general registry with the graphics on the inner surface of the film.

Other features and aspects of the present invention are discussed in greater detail below.

### BRIEF DESCRIPTION OF FIGURES

A full and enabling disclosure of the present invention, including the best mode thereof to one skilled in the art, is set forth more particularly in the remainder of the specification, which includes reference to the accompanying figures, in which:

FIG. 1 is a perspective view of an exemplary training pant 10; and

FIG. 2 is an exploded cross-sectional view of FIG. 1 taken along line 2-2.

FIGS. 3 a-c are cross-sectional views of different embodiments of sheet materials of the present invention.

FIG. 4 is a cross-sectional exploded view of an alternate sheet material according to the present invention.

FIG. 5 is a cross-sectional view of an alternate sheet material according to the present invention.

FIG. 6 is a top plan view of a softening agent-treated and printed film layer according to the present invention.

Repeat use of reference characters in the present specification and drawings is intended to represent the same or analogous features or elements of the present invention.

### DETAILED DESCRIPTION OF REPRESENTATIVE EMBODIMENTS

### Definitions

As used herein the term “nonwoven fabric or web” refers to a web having a structure of individual fibers or threads which are interlaid, but not in an identifiable manner as in a knitted fabric. Nonwoven fabrics or webs have been formed from many processes such as for example, meltblowing processes, spunbonding processes, bonded carded web processes, etc.

As used herein, the term “meltblown web” generally refers to a nonwoven web that is formed by a process in which a molten thermoplastic material is extruded through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “spunbond web” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. Nos. 4,340,563 to Appel, et al., 3,692,618 to Dorschner, et al., 3,802,817 to Matsuki, et al., 3,338,992 to Kinney, 3,341,394 to Kinney, 3,502,763 to Hartman, 3,502,538 to Levy, 3,542,615 to Dobo, et al., and 5,382,400 to Pike, et al., which are incorporated herein in their entirety by reference thereto for all purposes. Spunbond fibers are generally not tacky when they are deposited onto a collecting surface. Spunbond fibers may sometimes have diameters less than about 40 microns, and are often between about 5 to about 20 microns. To provide additional web integrity the webs so formed can be subjected to additional fiber bonding techniques if so desired.

As used herein, the term “coform” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic and/or organic absorbent materials, treated polymeric staple fibers and so forth. Some examples of such coform materials are disclosed in U.S. Pat. Nos. 4,100,324 to Anderson, et al.; 5,284,703 to Everhart, et al.; and 5,350,624 to Georger, et al.; which are incorporated herein in their entirety by reference thereto for all purposes.

As used herein, the term “multicomponent fibers” generally refers to fibers that have been formed from at least two polymer components. Such fibers are typically extruded from separate extruders, but spun together to form one fiber. The polymers of the respective components are typically different, but may also include separate components of similar or identical polymeric materials. The individual components are typically arranged in substantially constantly positioned distinct zones across the cross-section of the fiber and extend substantially along the entire length of the fiber. The configuration of such fibers may be, for example, a side-by-side arrangement, a pie arrangement, or any other arrangement. Multicomponent fibers and methods of making the same are taught in U.S. Pat. Nos. 5,108,820 to Kaneko, et al., 4,795,668 to Kruege, et al., 5,382,400 to Pike, et al., 5,336,552 to Strack, et al., and 6,200,669 to Marmon, et al., which are incorporated herein in their entirety by reference thereto for all purposes. The fibers and individual components containing the same may also have various irregular shapes such as those described in U.S. Pat. Nos. 5,277,976 to Hogle, et al., 5,162,074 to Hills, 5,466,410 to Hills, 5,069,970 to Largman, et al., and 5,057,368 to Largman, et al., which are incorporated herein in their entirety by reference thereto for all purposes.

As used herein, the terms “elastomeric” and “elastic” refer to a material that, upon application of a stretching force, is stretchable in at least one direction (such as the “MD” or machine direction or the “CD” or cross-machine direction), and which upon release of the stretching force, contracts/returns to approximately its original dimension. For example, a stretched material may have a stretched length that is at least 50% greater than its relaxed unstretched length, and which will recover to within at least 50% of its stretched length upon release of the stretching force. A hypothetical example would be a one (1) inch sample of a material that is stretchable to at least 1.50 inches and which, upon release of the stretching force, will recover to a length of not more than 1.25 inches. Desirably, such an elastomeric sheet contracts or recovers at least 50%, and even more desirably, at least 80% of the stretch length in the cross machine direction.

As used herein, the term “breathable” means pervious to water vapor and gases, but impermeable to liquid water. For instance, “breathable barriers” and “breathable films” allow water vapor to pass therethrough, but are substantially impervious to liquid water. The “breathability” of a material is measured in terms of water vapor transmission rate (WVTR), with higher values representing a more vapor-pervious material and lower values representing a less vapor-pervious material. Typically, the “breathable” materials have a water vapor transmission rate (WVTR) of from about 500 to about 20,000 grams per square meter per 24 hours (g/m²/24 hours), in some embodiments from about 1,000 to about 20,000, in some embodiments from about 1,000 to about 15,000 g/m²/24 hours, and in some embodiments, from about 1,500 to about 14,000 g/m²/24 hours.

As used herein, an “absorbent article” refers to any article capable of absorbing water or other fluids. Examples of some absorbent articles include, but are not limited to, personal care absorbent articles, such as diapers, training pants, absorbent underpants, adult incontinence products, feminine hygiene products (e.g., sanitary napkins), swim wear, baby wipes, and so forth; medical absorbent articles, such as garments, fenestration materials, underpads, bandages, absorbent drapes, and medical wipes; food service wipers; clothing articles; and so forth. Materials and processes suitable for forming such absorbent articles are well known to those skilled in the art.

As used herein, the term “hydrophobic substrate” is meant to include any shaped article, provided it is composed, in whole or in part, of a hydrophobic polymer and the term “porous hydrophobic substrate” is meant to include any substrate, provided it is porous and composed, in whole or in part, of a hydrophobic polymer. For example, the hydrophobic substrate may be a sheet-like material, such as a sheet of a foamed material. The hydrophobic substrate also may be a fibrous fabric, such as fibrillated film or a woven or nonwoven web or fabric. These structures can be predominately hydrophobic or can be selectively treated exhibiting different hydrophobic zones. Nonwoven fabrics include, but are not limited to, a meltblown fabric, a spunbonded fabric, a carded fabric or an airlaid fabric. The hydrophobic substrate also may be a laminate of two or more layers of a sheet-like material. For example, the layers may be independently selected from meltblown fabrics and spunbonded fabrics. However, other sheet-like materials such as films or foams may be used in addition to, or instead of or in combination with meltblown and spunbonded fabrics. In addition, the layers of the laminate may be prepared from the same hydrophobic polymer or different hydrophobic polymers.

The term “hydrophobic polymer” is used herein to mean any polymer resistant to wetting, or not readily wet, by water, i.e., having a lack of affinity for water. Examples of hydrophobic polymers include, by way of illustration only, polyolefins, such as polyethylene, poly(isobutene), poly(isoprene), poly(4-methyl-1-pentene), polypropylene, ethylene-propylene copolymers, ethylene-propylene-hexadiene copolymers, and ethylene-vinyl acetate copolymers; styrene polymers, such as poly(styrene), poly(2-methylstyrene), styrene-acrylonitrile copolymers having less than about 20 mol-percent acrylonitrile, and styrene-2,2,3,3,-tetrafluoropropyl methacrylate copolymers; halogenated hydrocarbon polymers, such as poly(chlorotrifluoroethylene), chlorotrifluoroethylene-tetrafluoroethylene copolymers, poly(hexafluoropropylene), poly(tetrafluoroethylene), tetrafluoroethylene-ethylene copolymers, poly(trifluoroethylene), poly(vinyl fluoride), and poly(vinylidene fluoride); vinyl polymers, such as poly(vinyl butyrate), poly(vinyl decanoate), poly(vinyl dodecanoate), poly(vinyl hexadecanoate), poly(vinyl hexanoate), poly(vinyl propionate), poly(vinyl octanoate), poly(heptafluoroisopropoxyethylene), poly(heptafluoroisopropoxypropylene), and poly(methacrylonitrile); acrylic polymers, such as poly(n-butyl acetate), poly(ethyl acrylate), poly[(1-chlorodifluoromethyl)tetrafluoroethyl acrylate], poly[di(chlorofluoromethyl)fluoromethyl acrylate], poly(1,1-dihydroheptafluorobutyl acrylate), poly(1,1-dihydropentafluoroisopropyl acrylate), poly(1,1-dihydropentadecafluorooctyl acrylate), poly(heptafluoroisopropyl acrylate), poly[5-(heptafluoroisopropoxy)pentyl acrylate], poly[11-(heptafluoroisopropoxy)undecyl acrylate], poly[2-(heptafluoropropoxy)ethyl acrylate], and poly(nonafluoroisobutyl acrylate); methacrylic polymers, such as poly(benzyl methacrylate), poly(n-butyl methacrylate), poly(isobutyl methacrylate), poly(t-butyl methacrylate), poly(t-butylaminoethyl methacrylate), poly(dodecyl methacrylate), poly(ethyl methacrylate), poly(2-ethylhexyl methacrylate), poly(n-hexyl methacrylate), poly(phenyl methacrylate), poly(n-propyl methacrylate), poly(octadecyl methacrylate), poly(1,1-dihydropentadecafluorooctyl methacrylate), poly(heptafluoroisopropyl methacrylate), poly(heptadecafluorooctyl methacrylate), poly(1-hydrotetrafluoroethyl methacrylate), poly(1,1-dihydrotetrafluoropropyl methacrylate), poly(1-hydrohexafluoroisopropyl methacrylate), and poly)ₜ-nonafluorobutyl methacrylate); and polyesters, such a poly(ethylene terephthalate) and poly(butylene terephthalate).

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. When ranges for parameters are given, it is intended that each of the endpoints of the range are also included within the given range. It is to be understood by one of ordinary skill in the art that the present discussion is a description of exemplary embodiments only, and is not intended as limiting the broader aspects of the present invention, which broader aspects are embodied exemplary constructions.

In one embodiment, the present disclosure is directed to a printed nonwoven web of synthetic fibers treated with an external softening agent. The web exhibits improved softness when a softening agent, also referred to as a softness treatment composition, is applied over a graphic on a visible outer surface of the nonwoven web. For example, the nonwoven web demonstrates improved softness when subjected to a static coefficient of friction test. As another example, the nonwoven web demonstrates improved softness when subjected to a dynamic coefficient of friction test. As a further example, the nonwoven web demonstrates improved softness without sacrificing any durability in that the nonwoven web demonstrates good durability when subjected to a crockfastness test.

In one embodiment, the printed and treated surface of the nonwoven web can be the visible surface of a laminate. For example, the printed and treated surface can be an outward facing surface (e.g., outer visible surface) of an outercover film-web laminate of an absorbent article. As such, the graphic and softness treatment composition can be applied directly onto the outer facing nonwoven surface of the outer cover, instead of on an underlying layer of the outer cover laminate (e.g., a film).

In another embodiment, the graphic can be applied to the innermost surface of the film layer and the softness treatment composition can be applied to just the exterior surface of the film layer or to the interior and exterior surfaces of both the nonwoven layer and the film layer. In these configurations, it has been found that the addition of the softening agent to the film layer reduces its opacity thereby making the underlying ink/graphics more visible while the treatment of the nonwoven layer provides improved softness.

In still another embodiment, the graphic can be applied to the surface of another layer that is subjacent to the inner surface of the film layer (and therefore closer to absorbent core of the product). In this case, from a visibility improvement standpoint, it may be desirable to apply a coating of the softening agent to any intervening surfaces between the graphics and the exterior of the film-web laminate.

To improve the visibility of the graphics, the softening agent can be applied to the entire surface of a film layer or it can be applied to discrete locations of the film layer on the side opposite the graphics such that the softening agent in applied in vertical juxtaposition to the graphics and thus the graphics and softening agent are in general registry with one another. In this way, the cost of the softening agent is kept to a minimum as it is not wasted on areas of the film where improved visibility is not necessary since there are no graphics directly underlying the untreated areas of the film layer.

### A. Substrates

The substrates to which the graphic and softening treatment may be applied include any known sheet-like substrate, such as films, nonwoven webs (e.g., spunbond webs, meltblown webs, and so forth), etc. The substrate may contain a single layer or multiple layers and may also contain additional materials such that it is considered a composite. In one embodiment, the substrate may be a nonwoven web of synthetic fibers. The synthetic fibers can generally be hydrophobic fibers. In one particular embodiment, the fibers of the nonwoven web are primarily hydrophobic synthetic fibers. For example, greater than about 90% of the fibers of the web can be hydrophobic synthetic fibers, such as greater than about 95%. In one embodiment, substantially all of the fibers of the nonwoven web (i.e., greater than about 98%, greater than about 99%, or about 100%) are hydrophobic synthetic fibers.

The nonwoven web can be made by any number of processes. As a practical matter, however, the nonwoven fabrics and the fibers that make up nonwoven fabrics usually will be prepared by a melt-extrusion process and formed into the nonwoven fabric. The term melt-extrusion process includes, among others, such well-known processes as meltblowing and spunbonding. Other methods for preparing nonwoven fabrics are, of course, known and may be employed. Such methods include air laying, wet laying, carding, and so forth. In some cases it may be either desirable or necessary to stabilize the nonwoven fabric by known means, such as thermal point bonding, through-air bonding, and hydroentangling.

As stated, the nonwoven web can primarily include synthetic fibers, particularly synthetic hydrophobic fibers, such as polyolefin fibers. In one particular embodiment, polypropylene fibers can be used to form the nonwoven web. The polypropylene fibers as with other polymer fibers may have a denier per filament of about 1.5 to 2.5, and the nonwoven web may have a basis weight of about 17 grams per square meter (0.5 ounce per square yard) and may range from about 10 grams per square meter to about 30 grams per square meter, however, other basis weights and ranges may be appropriate depending upon the particular end use. Note, however, that other fiber types and sizes as well as types of nonwoven webs and basis weights may be used depending upon the particular end use application. Furthermore, the nonwoven fabric may include bicomponent or other multicomponent fibers. Exemplary multicomponent nonwoven fabrics are described in U.S. Pat. No. 5,382,400 issued to Pike et al., U.S. Publication no. 2003/0118816 entitled “High Loft Low Density Nonwoven Fabrics Of Crimped Filaments And Methods Of Making Same” and U.S. Publication no. 2003/0203162 entitled “Methods For Making Nonwoven Materials On A Surface Having Surface Features And Nonwoven Materials Having Surface Features” which are hereby incorporated by reference herein in their entirety.

Sheath/core bicomponent fibers where the sheath is a polyolefin such as polyethylene or polypropylene and the core is polyester such as poly(ethylene terephthalate) or poly(butylene terephthalate) can also be used to produce carded fabrics or spunbonded fabrics. The primary role of the polyester core is to provide resiliency and thus to maintain or recover bulk under/after load. Bulk retention and recovery plays a role in separation of the skin from the absorbent structure. This separation has shown an effect on skin dryness. The combination of skin separation provided with a resilient structure along with a treatment such of the present invention can provide an overall more efficient material for fluid handling and skin dryness purposes.

If desired, a treatment composition can be applied to the substrate prior to application of the ink composition to further adhere the ink composition to the nonwoven web. For example, a surface treatment can comprise a polyurethane alone or in combination with a cationic species, such as a cationic polymer. In some embodiments, the surface treatment can also include other additives, such as inorganic particles, organic particles, surfactants, pH modifiers, crosslinkers, binders, and any combination or mixture thereof. It also can comprise a coating or other surface treatment of a polysaccharide, a modified polysaccharide, a derivative of a polysaccharide, or a derivative of a modified polysaccharide. Exemplary treatment compositions that can be utilized are disclosed in U.S. Publication No. 2004/0121675 to Snowden, et al., U.S. Publication No. 2006/0003150 to Braverman, et al., and U.S. Publication No. 2006/0246263 to Yahiaoui, et al., all of which are herein incorporated by reference.

As stated, the nonwoven web can be included as an outer surface of a laminate. When included as part of a laminate, the nonwoven web generally provides a more cloth-like feeling to the laminate. For example, a film-web laminate can be formed from the nonwoven web overlying a film layer. In one embodiment, for instance, the nonwoven web is thermally laminated to the film to form the film-web laminate. In other instances, the nonwoven web can be adhesively or ultrasonically laminated to the film to form the film-web laminate. However, any suitable technique can be utilized to form the laminate. Suitable techniques for bonding a film to a nonwoven web are described in U.S. Pat. Nos. 5,843,057 to McCormack; 5,855,999 to McCormack; 6,002,064 to Kobylivker, et al.; 6,037,281 to Mathis, et al.; and WO 99/12734, which are incorporated herein in their entirety by reference thereto for all purposes.

The film layer of the laminate is typically formed from a material that is substantially impermeable to liquids. For example, the film layer may be formed from a thin plastic film or other flexible liquid-impermeable material. In one embodiment, the film layer is formed from a polyethylene or other polymer film having a thickness of from about 0.01 millimeter to about 0.05 millimeter. For example, a stretch-thinned polypropylene or other polymer film having a thickness of about 0.015 millimeter may be thermally laminated to the nonwoven web.

In addition, the film layer may be formed from a material that is impermeable to liquids, but permeable to gases and water vapor (i.e., “breathable”). This permits vapors to pass through the laminate, but still prevents liquid exudates from passing through the laminate. The use of a breathable laminate is especially advantageous when the laminate is used as an outercover of an absorbent article to permit vapors to escape from the absorbent core, but still prevents liquid exudates from passing through the outer cover. For example, the breathable film may be a microporous or monolithic film.

The film may be formed from a polyolefin polymer, such as linear, low-density polyethylene (LLDPE) or polypropylene. Examples of predominately linear polyolefin polymers include, without limitation, polymers produced from the following monomers: ethylene, propylene, 1-butene, 4-methyl-pentene, 1-hexene, 1-octene and higher olefins as well as copolymers and terpolymers of the foregoing. In addition, copolymers of ethylene and other olefins including butene, 4-methyl-pentene, hexene, heptene, octene, decene, etc., are also examples of predominately linear polyolefin polymers.

If desired, the breathable film may also contain an elastomeric polymer, such as elastomeric polyesters, elastomeric polyurethanes, elastomeric polyamides, elastomeric polyolefins, elastomeric copolymers, and so forth. Examples of elastomeric copolymers include block copolymers having the general formula A-B-A′ or A-B, wherein A and A′ are each a thermoplastic polymer endblock that contains a styrenic moiety (e.g., poly(vinyl arene)) and wherein B is an elastomeric polymer midblock, such as a conjugated diene or a lower alkene polymer (e.g., polystyrene-poly(ethylene-butylene)-polystyrene block copolymers). Also suitable are polymers composed of an A-B-A-B tetrablock copolymer, such as discussed in U.S. Pat. No. 5,332,613 to Taylor, et al., which is incorporated herein in its entirety by reference thereto for all purposes. An example of such a tetrablock copolymer is a styrene-poly(ethylene-propylene)-styrene-poly(ethylene-propylene) (“S-EP-S-EP”) block copolymer. Commercially available A-B-A′ and A-B-A-B copolymers include several different formulations from Kraton Polymers of Houston, Tex. under the trade designation KRATON®. KRATON® block copolymers are available in several different formulations, a number of which are identified in U.S. Pat. Nos. 4,663,220, 4,323,534, 4,834,738, 5,093,422 and 5,304,599, which are hereby incorporated in their entirety by reference thereto for all purposes. Other commercially available block copolymers include the S-EP-S or styrene-poly(ethylene-propylene)-styrene elastomeric copolymer available from Kuraray Company, Ltd. of Okayama, Japan, under the trade name SEPTON®.

Examples of elastomeric polyolefins include ultra-low density elastomeric polypropylenes and polyethylenes, such as those produced by “single-site” or “metallocene” catalysis methods. Such elastomeric olefin polymers are commercially available from ExxonMobil Chemical Co. of Houston, Tex. under the trade designations ACHIEVE® (propylene-based), EXACT® (ethylene-based), and EXCEED® (ethylene-based). Elastomeric olefin polymers are also commercially available from DuPont Dow Elastomers, LLC (a joint venture between DuPont and the Dow Chemical Co.) under the trade designation ENGAGE® (ethylene-based) and AFFINITY® (ethylene-based). Examples of such polymers are also described in U.S. Pat. Nos. 5,278,272 and 5,272,236 to Lai, et al., which are incorporated herein in their entirety by reference thereto for all purposes. Also useful are certain elastomeric polypropylenes, such as described in U.S. Pat. Nos. 5,539,056 to Yang, et al. and 5,596,052 to Resconi, et al., which are incorporated herein in their entirety by reference thereto for all purposes.

If desired, blends of two or more polymers may also be utilized to form the breathable film. For example, the film may be formed from a blend of a high performance elastomer and a lower performance elastomer. A high performance elastomer is generally an elastomer having a low level of hysteresis, such as less than about 75%, and in some embodiments, less than about 60%. Likewise, a low performance elastomer is generally an elastomer having a high level of hysteresis, such as greater than about 75%. The hysteresis value may be determined by first elongating a sample to an ultimate elongation of 50% and then allowing the sample to retract to an amount where the amount of resistance is zero. Particularly suitable high performance elastomers may include styrenic-based block copolymers, such as described above and commercially available from Kraton Polymers of Houston, Tex. under the trade designation KRATON®. Likewise, particularly suitable low performance elastomers include elastomeric polyolefins, such as metallocene-catalyzed polyolefins (e.g., single site metallocene-catalyzed linear low density polyethylene) commercially available from DuPont Dow Elastomers, LLC under the trade designation AFFINITY®. In some embodiments, the high performance elastomer may constitute from about 25 wt. % to about 90 wt. % of the polymer component of the film, and the low performance elastomer may likewise constitute from about 10 wt. % to about 75 wt. % of the polymer component of the film. Further examples of such a high performance/low performance elastomer blend are described in U.S. Pat. No. 6,794,024 to Walton, et al., which is incorporated herein in its entirety by reference thereto for all purposes.

As stated, the breathable film may be microporous. The micropores form what is often referred to as tortuous pathways through the film. Liquid contacting one side of the film does not have a direct passage through the film. Instead, a network of microporous channels in the film prevents liquids from passing, but allows gases and water vapor to pass. Microporous films may be formed from a polymer and a filler (e.g., calcium carbonate). Fillers are particulates or other forms of material that may be added to the film polymer extrusion blend and that will not chemically interfere with the extruded film, but which may be uniformly dispersed throughout the film. Generally, on a dry weight basis, based on the total weight of the film, the film includes from about 30% to about 90% by weight of a polymer. In some embodiments, the film includes from about 30% to about 90% by weight of a filler. Examples of such films are described in U.S. Pat. Nos. 5,843,057 to McCormack; 5,855,999 to McCormack; 5,932,497 to Morman, et al.; 5,997,981 to McCormack et al.; 6,002,064 to Kobylivker, et al.; 6,015,764 to McCormack, et al.; 6,037,281 to Mathis, et al.; 6,111,163 to McCormack, et al.; and 6,461,457 to Taylor, et al., which are incorporated herein in their entirety by reference thereto for all purposes.

The films are generally made breathable by stretching the filled films to create the microporous passageways as the polymer breaks away from the filler (e.g., calcium carbonate) during stretching. For example, the breathable material contains a stretch-thinned film that includes at least two basic components, i.e., a polyolefin polymer and filler. These components are mixed together, heated, and then extruded into a film layer using any one of a variety of film-producing processes known to those of ordinary skill in the film processing art. Such film-making processes include, for example, cast embossed, chill and flat cast, and blown film processes.

Another type of breathable film is a monolithic film that is a nonporous, continuous film, which because of its molecular structure, is capable of forming a liquid-impermeable, vapor-permeable barrier. Among the various polymeric films that fall into this type include films made from a sufficient amount of poly(vinyl alcohol), polyvinyl acetate, ethylene vinyl alcohol, polyurethane, ethylene methyl acrylate, and ethylene methyl acrylic acid to make them breathable. Without intending to be held to a particular mechanism of operation, it is believed that films made from such polymers solubilize water molecules and allow transportation of those molecules from one surface of the film to the other. Accordingly, these films may be sufficiently continuous, i.e., nonporous, to make them substantially liquid-impermeable, but still allow for vapor permeability.

Breathable films, such as described above, may constitute the entire breathable material, or may be part of a multilayer film. Multilayer films may be prepared by cast or blown film coextrusion of the layers, by extrusion coating, or by any conventional layering process. Further, other breathable materials that may be suitable for use in the present invention are described in U.S. Pat. Nos. 4,341,216 to Obenour; 4,758,239 to Yeo, et al.; 5,628,737 to Dobrin, et al.; 5,836,932 to Buell; 6,114,024 to Forte; 6,153,209 to Vega, et al.; 6,198,018 to Curro; 6,203,810 to Alemany, et al.; and 6,245,401 to Ying, et al., which are incorporated herein in their entirety by reference thereto for all purposes.

In one embodiment, the laminate consists only of two layers: the nonwoven web and the film. On the other hand, in some embodiments, other layers may be included in the laminate, so long as the nonwoven web defines an outer surface of the laminate which, in certain embodiments, is capable of receiving the ink composition and the softening agent. When present, the other layer(s) of the laminate can include, nonwoven webs, films, foams, etc.

In one particular embodiment, the nonwoven web is suitable for use as a layer of a backsheet laminate (i.e., outercover) of an absorbent article. The backsheet of absorbent articles is typically a liquid impermeable sheet, and may also be breathable. For example, in one particular embodiment, the backsheet is a laminate of a liquid impervious film attached to a nonwoven web of polyolefin fibers.

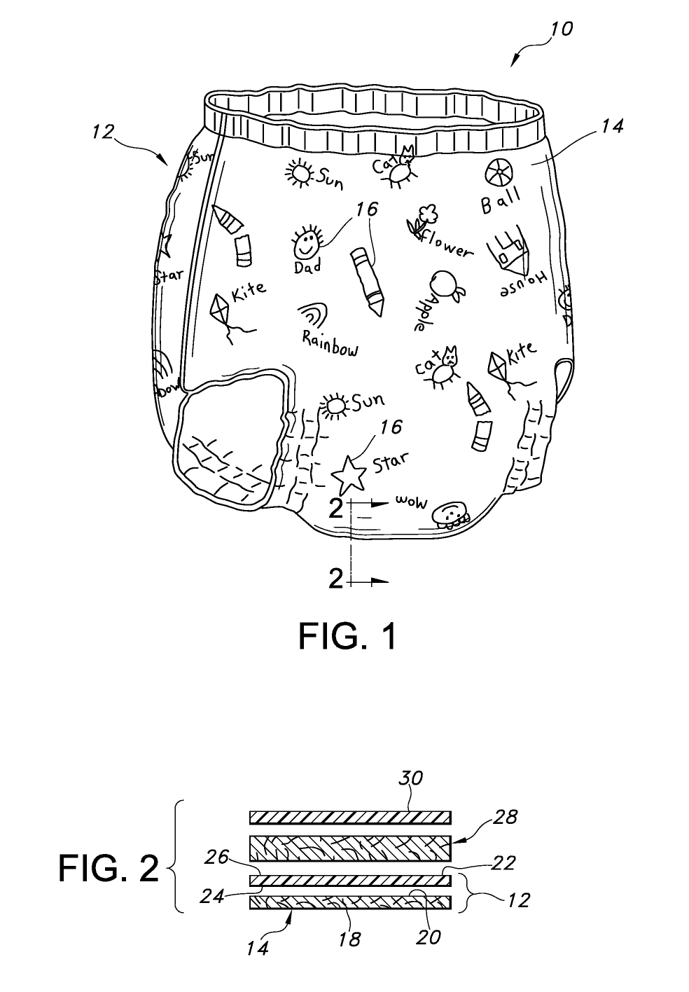

An exemplary printed nonwoven web laminate used in the construction of an exemplary training pant is illustrated in FIGS. 1, 2, and 3 a-c. FIG. 1 is a perspective view of an exemplary training pant 10, and FIG. 2 is an exploded cross-sectional view of FIG. 1 taken along line 2-2. It is an outer visible surface 18 of a nonwoven web 14 that presents or forms the outermost, visible surface of a training pant 10 and on which images 16 can be printed. The illustrated exemplary printed nonwoven web 14 is utilized as the outward facing layer of a backsheet 12 of the training pant 10, but could be incorporated on any of a variety of absorbent articles upon which printed information or designs might be desirable including, but not limited to, diapers, feminine care products, incontinence products, training pants, swimming pants, and so forth.

For example, in FIGS. 1 and 2, the training pant 10 comprises the backsheet 12, which can be a two-layered laminate that includes a nonwoven polyolefin fibrous web 14 suitably joined to a liquid impervious film 22. The web 14 has opposed surfaces such as an inner surface 20 and an outer visible surface 18. A film 22 has opposed surfaces such as an outer surface 24 facing an inner surface 20 of the web 14 and an inner surface 26 that faces toward an absorbent composite 28.

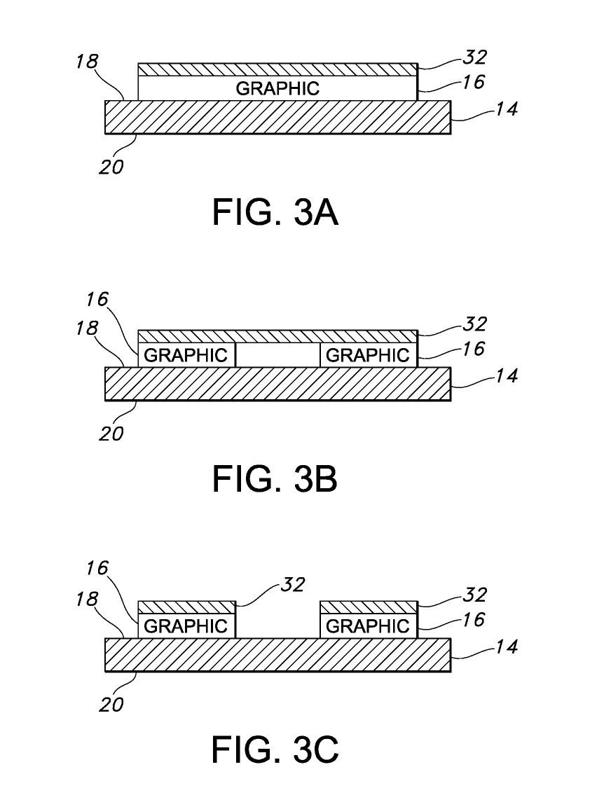

One way to make these products more appealing is to print in bright colors on the products. FIGS. 3 a-c depict cross-sectional views of a several configurations of printed nonwoven webs 14. Any desired design or other image 16 can be applied or printed on the outer visible surface 18 defined by the nonwoven web 14 using a suitable ink composition. For example, a number of intricate, registered images 16 can be printed on the outer visible surface 18 of the backsheet 12. A softening agent 32 is then applied over the ink composition that forms the graphical image 16. By outer “visible” surface is meant that surface of the product that is visible when the product is worn (i.e., the exposed outer surface of the absorbent article). As stated, the inclusion of a crosslinking agent in the ink composition can inhibit the design from rubbing off during use of the article. As such, the designs can resist fading on the outercover or backsheet 12, as well as preventing staining of anything (e.g., skin) that contacts the outercover. In the embodiment depicted in FIG. 3a, both the graphic image 16 and the softening agent 32 are shown extending across the outer visible surface 18 of the nonwoven 14. In another embodiment depicted in FIG. 3b, discrete graphics 16 are depicted on the outer visible surface 18 of the nonwoven 14. The softening agent 32 extends from one discrete graphic 16 to the next as the softening agent 32 extends across the outer visible surface 18. In a further embodiment depicted in FIG. 3c, discrete graphics 16 are depicted on the outer visible surface 18 of the nonwoven 14 with discrete areas of softening agent 32 applied in a pattern similar to that of the discrete graphics 16.

A liquid permeable topsheet 30 (i.e., the bodyside liner) is positioned on the side of the absorbent core 28 opposite to the backsheet 12, and is the layer that is against the skin of the wearer.

The liquid permeable topsheet 30 is generally employed to help isolate the wearer's skin from liquids held in the absorbent core 28. For example, the liquid permeable topsheet 30 presents a bodyfacing surface that is typically compliant, soft feeling, and non-irritating to the wearer's skin. Typically, the liquid permeable topsheet 30 is also less hydrophilic than the absorbent core 28 so that its surface remains relatively dry to the wearer. The liquid permeable topsheet 30 permits liquid to readily penetrate through its thickness.

The liquid permeable topsheet 30 may be formed from a wide variety of materials, such as porous foams, reticulated foams, apertured plastic films, natural fibers (e.g., wood or cotton fibers), synthetic fibers (e.g., polyester or polypropylene fibers), or a combination thereof. In some embodiments, woven and/or nonwoven fabrics are used for the liquid permeable topsheet 30. For example, the liquid permeable topsheet 30 may be formed from a meltblown or spunbonded web of polyolefin fibers. The liquid permeable topsheet 30 may also be a bonded-carded web of natural and/or synthetic fibers. The liquid permeable topsheet 30 may further be composed of a substantially hydrophobic material that is optionally treated with a surfactant or otherwise processed to impart a desired level of wettability and hydrophilicity. The surfactant may be applied by any conventional method, such as spraying, printing, brush coating, foaming, and so forth. When utilized, the surfactant may be applied to the entire liquid permeable topsheet 30 or may be selectively applied to particular sections of the liquid permeable topsheet 30, such as to the medial section along the longitudinal centerline of the diaper. The liquid permeable topsheet 30 may further include a composition that is configured to transfer to the wearer's skin for improving skin health. Suitable compositions for use on the liquid permeable topsheet 30 are described in U.S. Pat. No. 6,149,934 to Krzysik et al., which is incorporated herein in its entirety by reference thereto for all purposes.

The absorbent core 28 may be formed from a variety of materials, but typically includes a matrix of hydrophilic fibers. In one embodiment, an absorbent web is employed that contains a matrix of cellulosic fluff fibers. One type of fluff that may be used in the present invention is identified with the trade designation CR1654, available from U.S. Alliance of Childersburg, Ala., and is a bleached, highly absorbent sulfate wood pulp containing primarily softwood fibers. Airlaid webs may also be used. In an airlaying process, bundles of small fibers having typical lengths ranging from about 3 to about 19 millimeters are separated and entrained in an air supply and then deposited onto a forming screen, usually with the assistance of a vacuum supply. The randomly deposited fibers then are bonded to one another using, for example, hot air or a spray adhesive. Another type of suitable absorbent nonwoven web for the absorbent core 28 is a coform material, which may be a blend of cellulose fibers and meltblown fibers.

In some embodiments, the absorbent core 28 may contain a superabsorbent material, e.g., a water-swellable material capable of absorbing at least about 20 times its weight and, in some cases, at least about 30 times its weight in an aqueous solution containing 0.9 weight percent sodium chloride. The superabsorbent materials may be natural, synthetic and modified natural polymers and materials. In addition, the superabsorbent materials may be inorganic materials, such as silica gels, or organic compounds such as cross-linked polymers. Examples of synthetic superabsorbent material polymers include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), polyacrylamido-methyl propane sulfonic acid and salt, and mixtures and copolymers thereof. Further superabsorbent materials include natural and modified natural polymers, such as hydrolyzed acrylonitrile-grafted starch, acrylic acid grafted starch, methyl cellulose, chitosan, carboxymethyl cellulose, hydroxypropyl cellulose, and the natural gums, such as alginates, xanthan gum, locust bean gum and so forth. Mixtures of natural and wholly or partially synthetic superabsorbent polymers may also be useful in the present invention. Other suitable absorbent gelling materials are disclosed in U.S. Pat. Nos. 3,901,236 to Assarsson et al.; 4,076,663 to Masuda et al.; and 4,286,082 to Tsubakimoto et al., which are incorporated herein in their entirety by reference thereto for all purposes.

Although not specifically illustrated, absorbent articles, such as the exemplary training pant shown in FIG. 1, may also include other layers not illustrated. For example, a surge layer can be included in the construction of the article to help decelerate and diffuse surges or gushes of liquid that may be rapidly introduced into the absorbent core 28. Desirably, the surge layer can rapidly accept and temporarily hold the liquid prior to releasing it into the storage or retention portions of the absorbent core 28. Typically, when included in the article, the surge layer is interposed between liquid permeable topsheet and the absorbent core. Alternatively, the surge layer may be located on an outwardly facing surface of the liquid permeable topsheet. The surge layer is typically constructed from highly liquid-permeable materials. Suitable materials may include porous woven materials, porous nonwoven materials, and apertured films. Some examples include, without limitation, flexible porous sheets of polyolefin fibers, such as polypropylene, polyethylene or polyester fibers; webs of spunbonded polypropylene, polyethylene or polyester fibers; webs of rayon fibers; bonded carded webs of synthetic or natural fibers or combinations thereof. Other examples of suitable surge layers are described in U.S. Pat. No. 5,486,166 to Ellis, et al. and 5,490,846 to Ellis, et al., which are incorporated herein in their entirety by reference thereto for all purposes.

Besides the above-mentioned components, absorbent articles may also contain various other components as is known in the art. For example, the absorbent article may also contain a substantially hydrophilic tissue wrapsheet (not illustrated) that helps maintain the integrity of the airlaid fibrous structure of the absorbent core 28. The tissue wrapsheet is typically placed about the absorbent core over at least the two major facing surfaces thereof, and can be composed of an absorbent cellulosic material, such as creped wadding or a high wet-strength tissue. The tissue wrapsheet may be configured to provide a wicking layer that helps to rapidly distribute liquid over the mass of absorbent fibers of the absorbent core. The wrapsheet material on one side of the absorbent fibrous mass may be bonded to the wrapsheet located on the opposite side of the fibrous mass to effectively entrap the absorbent core. Alternatively, the wrapsheet can be made from a meltblown fibrous web which can be treated with a surfactant or other wetting agent to make it more hydrophilic. More detailed descriptions of training pants can be found in U.S. Pat. No. 4,940,464 to Van Gompel et al., the entire contents of which are hereby incorporated by reference herein.

### B. Ink Compositions

Ink compositions can be applied in a solution form, such as in an aqueous solution, an organic solvent solution, or in mixed aqueous/organic solvent systems. Typically, aqueous based ink compositions are most widely used with digital printing, while solvent based inks are most widely used with flexographic printing. However, solvent based inks can also be used with digital processes, and water based inks are commonly used with flexographic printing. In the digital ink processes, the inks are difficult to formulate because the ink composition is constrained to a choice of ingredients. The digital inks and digital processes have narrow tolerances in terms of pH, viscosity, surface tension, purity, and other physical and chemical properties. Also, ink compositions used in digital ink processes typically have a low solid content which can create difficulty in drying the ink composition onto the polymeric substrate, especially in the high speed printing process.

In some embodiments, the ink composition may contain a crosslinking agent in an amount sufficient to crosslink molecules within the ink composition. As such, the ink composition becomes more coherent once applied to the substrate. For example, the ink composition can crosslink to form a 3-dimensional chemical structure once applied to the substrate. The 3-dimensional structure of the ink composition can inhibit the ink composition from rubbing off of the substrate through mechanical forces. For example, when applied to a nonwoven fabric, the ink composition can form a crosslinked structure that wraps around the fibers of the nonwoven fabric, effectively inhibiting the crosslinked ink composition from rubbing off of the fibers.

In other embodiments described in more detail below, the inks may be used to apply the graphics 16 to the inner surface 26 of the film layer 22. In this configuration, the ink and resultant graphics 16 can be more protected from external frictional forces encountered during use. Nonetheless, it still may be desirable to include the crosslinking agent in the ink composition.

Additionally, the crosslinking agent may crosslink molecules within the ink composition to suitable sites on the substrate itself. Thus, the ink composition can be chemically bonded to the substrate and inhibited from rubbing off of the fibers or film surface through chemical forces. For instance, when applied to a nonwoven web, the molecules within the ink composition can bond to polymers within the fibers of the nonwoven web and to the surface of polymeric films.

Adding a relatively high amount of the crosslinking agent to the ink composition dramatically increases the oil crockfastness of the ink composition applied to the substrate. For example, the crosslinking agent can be added to the ink composition in an amount of greater than about 2% by weight based on the wet weight of the ink composition, such as greater than about 4% by weight. In some embodiments, the crosslinking agent can be present in an amount of from about 5% by weight to about 20% by weight, such as from about 7% by weight to about 15% by weight. For example, in one particular embodiment, the crosslinking agent can be present at about 10% to about 12% by weight in the ink composition. It should be noted that after application of the ink composition to the laminate, the dried ink composition may contain a greater percentage by dry weight of the crosslinking agent due to the solvent evaporating.

The crosslinking agent can be selected from those agents configured to crosslink the ink composition to form a three dimensional chemical structure. Additionally, the crosslinking agent can facilitate bonding between the ink composition and the fibers of the nonwoven web or the surface of the film. Examples of suitable crosslinking agents that may be used include, but are not limited to, XAMA-2, XAMA-7, and CX-100, which are available commercially from Noveon, Inc. of Cleveland, Ohio. These materials are aziridine oligomers with at least two aziridine functional groups. More detailed descriptions of ink compositions can be found in U.S. Patent Application No. 2008/0227356 to Poruthoor et al., the entire contents of which are hereby incorporated by reference herein.

Additionally, other adhesion promoters can be added to the ink composition. For example, Carboset 514H, available commercially from Noveon, Inc. of Cleveland, Ohio, is an acrylic colloidal dispersion polymer supplied in ammonia water, which can dry to a clear, water-resistant, non-tacky thermoplastic film.

In addition to the crosslinking agent, the ink compositions can generally contain a coloring agent (e.g., pigment or dye), a solvent, and any other desired ingredients. Typically, a pigment refers to a colorant based on inorganic or organic particles which do not dissolve in water or solvents. Usually pigments form an emulsion or a suspension in water. On the other hand, a dye generally refers to a colorant that is soluble in water or solvents.

The pigment or dye in the ink composition can be present in an amount effective to provide a visible mark once applied to the substrate. For example, the pigment or dye can be present in the ink composition at concentration between about 0.25% to about 40% based on the dry weight basis, and preferably between greater than or equal to about 1% and less than or equal to about 10%.

Suitable organic pigments, include dairylide yellow AAOT (for example, Pigment Yellow 14 CI No. 21 095), dairylide yellow AAOA (for example, Pigment Yellow 12 CI No. 21090), Hansa Yellow, CI Pigment Yellow 74, Phthalocyanine Blue (for example, Pigment Blue 15), lithol red (for example, Pigment Red 52:1 CI No. 15860:1). toluidine red (for example. Pigment Red 22 CI No. 12315), dioxazine violet (for example, Pigment Violet 23 CI No. 51319), phthalocyanine green (for example, Pigment Green 7 CI No. 74260), phthalocyanine blue (for example, Pigment Blue 15 CI No. 74160), naphthoic acid red (for example, Pigment Red 48:2 CI No. 15865:2). Inorganic pigments include titanium dioxide (for example, Pigment White 6 CI No. 77891), carbon black (for example, Pigment Black 7 CI No. 77266), iron oxides (for example, red, yellow. and brown), ferric oxide black (for example, Pigment Black 11 CI No. 77499), chromium oxide (for example, green), ferric ammonium ferrocyanide (for example, blue), and the like.

Suitable dyes that may be used with the additive of the present invention include, for instance, acid dyes, and sulfonated dyes including direct dyes. Other suitable dyes include azo dyes (e.g., Solvent Yellow 14, Dispersed Yellow 23, and Metanil Yellow), anthraquinone dyes (e.g., Solvent Red 111, Dispersed Violet 1, Solvent Blue 56, and Solvent Orange 3), xanthene dyes (e.g., Solvent Green 4, Acid Red 52, Basic Red 1, and Solvent Orange 63), azine dyes (e.g., Jet Black), and the like.

The inks are generally dispersed or dissolved in a low viscosity carrier such as a solvent. Exemplary solvents are aliphatic hydrocarbons with common binder types, such as polyamide, shellac, nitro-cellulose, and styrene-maleic. Generally, solvent-based inks include non-catalytic, block urethane resin, which generally demonstrate superior durability over traditional flexographic binders, such as styrene-maleic, rosin-maleic, and acrylic solutions. Desired solvent blends include various acetates such as ethyl acetate, N-propyl acetate, isopropyl acetate, isobutyl acetate, N-butyl acetate, and blends thereof; various alcohols including ethyl alcohol, isopropyl alcohol, normal propyl alcohol, and blends thereof; and glycol ethers including Ektasolve® EP (ethylene glycol monopropyl ether), EB (ethylene glycol monobutyl ether), DM (diethylene glycol monomethyl ether), DP (diethylene glycol monopropyl ether), and PM (propylene glycol monomethyl ether), which may be obtained from Eastman Chemical of Kingsport, Tenn. Other glycols that may also be used are DOWANOL® obtainable from Dow Chemical of Midland, Mich. A desired solvent blend may be a blend of about 50 percent to about 75 percent glycol ether, about 25 percent to about 35 percent N-propyl acetate, and about 15 percent to about 25 percent N-butyl acetate.

Suitable water-based inks that may be used include emulsions that may be stabilized in water-ammonia and may further comprise alcohols, glycols, or glycol ethers as co-solvents. If water-based inks are used on the underside of the film layer where they may be exposed to liquids such as body exudates, it may be desirable to crosslink the inks unless they are specialty inks such as those used for disappearing graphics which are commonly used as wetness indicators. Generally, organic solvents (less than equal to about 7 percent) such as for example, propan-2-ol, mono propylene glycol, glycol ethers, dipropyl glycol mono methyl ether and others may be added to water-based inks for assisting wetting, film formation and drying. Such solvents may be commodity chemicals, commercially available from various companies. Generally, water-based ink includes self-crosslinking acrylic copolymer emulsion, which may have demonstrated superior durability over traditional non-crosslinking binders such as acrylic solutions and dispersion copolymers. Besides the solvent and pigments, the inks may comprise a binder or mixtures thereof. The binder helps stabilize the pigment onto the cover layer or backsheet 12 or when printing on the inner surface 26 of the film layer 22. Generally, the pigment-to-binder ratios are typically from 1:20 to 1:2. Additionally, the film 22 and or laminate 12 may be treated with corona or plasma to enhance ink adhesion to the surface of the substrate.

Waxes may also be included in the ink composition to increase the slip and improve the rub-resistance of the inks of the printed polyolefin substrate. Common classifications of waxes include animal (for example, beeswax and lanolin), vegetable (for example, carnauba and candellilia), mineral (for example, paraffin and microcrystalline), and synthetic (for example, polyethylene, polyethylene glycol, and Teflon® polymers). In one embodiment, a wax can be present in an amount of about 0.5 percent to about 5 percent based on the total ink formulation weight when wet.

In one embodiment, the ink compositions used in the printing process to form the indicia are particulate-type ink compositions. The inks chosen should, of course, be safe for human use and should not have environmentally deleterious effects. Moreover, it is desirable that the ink composition is suitable for the intended printing process and is preferably temperature resistant to the process employed for forming the absorbent article, e.g., the temperatures used during a vacuum aperturing process and other elevated heating processes.

The particular method of printing the ink composition onto the nonwoven web 14 or the film layer 22 can be any suitable printing method, including flexographic, gravure, offset, ink-jet, etc. The printing method can be used to print any design, figure, or other image on the surface of the nonwoven web 14 or the film layer 22.

As well known in the art, each printing process generally requires a specific ink composition specially formulated for that particular printing process. The particular ink formulations generally compensate for differing printing conditions between different printing processes and differing print substrates. For example, ink compositions for ink-jet printing are considerably different than ink compositions for flexographic printing due in part to different types of printing systems used in the two processes.

For instance, flexographic inks are not limited by the type of coloring agent and can utilize dyes and/or pigments, even those pigments that contain a relatively large particle size. However, ink jet printing inks are generally limited to particle-free ink compositions, or at least those with a relatively small particle size. As such, ink jet inks typically include dyes as opposed to pigments as the coloring agent in the ink composition.

### C. Softening Agents

The softening agents used herein can serve one or both of two functions. The first function is to improve the softness of the outer visible surface 18 of the nonwoven web 14. Improvements in this regard can be demonstrated by improvements in the coefficient of friction of the outer visible surface 18 of the fibrous web 14. The second function of the softening agent 32 is to improve the transparency of the film layer 22 when the ink and graphics 16 are printed on the inner surface 26 of the film layer 22 so the visibility and clarity of the film layer 22 is improved. Improvements in the visibility and clarity can be demonstrated by improvements in the optical density and/or the opacity of the film-web laminate 12.

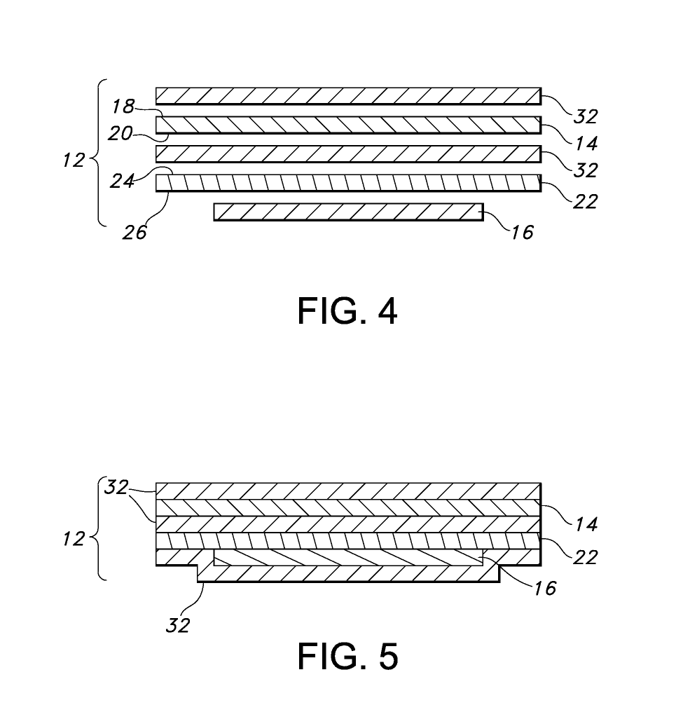

In FIGS. 3a through 3c, the substrate further includes a softening agent 32 overlaying the ink composition 16 on the outer visible surface 18 of the web 14 and thus the overall product 10. In FIGS. 4 through 6, the ink composition 16 is located on the inner surface 26 of the film layer 22.

Referring to FIGS. 3a through 3c, the softening agent may be present in a continuous layer over the entire external surface of the printed substrate. Alternatively, the softening agent may be present in a pattern on the printed external surface of the printed surface. In one embodiment, the softening agent is present on the printed surface of the nonwoven in a pattern that coincides with a printed pattern on the surface of the nonwoven. Desirably the softening agent is transparent or substantially transparent to permit the underlying printed pattern or graphic to be visible through the softening agent.

Referring to FIGS. 4 through 6, for purposes of improving the transparency and thus the resultant visibility and clarity of the applied ink composition/graphics 16, the softening agent 32 may be present in a continuous layer over the entire outer surface 24 of the printed film substrate 22. Alternatively, the softening agent 32 may be present in a pattern on the outer surface 24 of the film 22 that generally only coincides with a printed pattern 16 on the inner surface 26 of the printed film substrate 22 so that other areas of the printed film substrate 22 and optionally the laminate 12 overall are not treated with the softening agent 32 used to improve the transparency of the film substrate 22. Desirably the softening agent is transparent or substantially transparent to permit the underlying printed pattern or graphic 26 to be visible through the softening agent 32. Alternatively, the softening agent 32 may be applied to all surfaces (18, 20, 24 and 26) of both the fibrous nonwoven layer 14 and the film layer 22.

Suitably, when the softening agent 32 is being primarily used to improve the softness of the visible outer surface 18 of the fibrous nonwoven web 14, the softening agent 32 may include erucamide, cetyl 2-ethylhexanone, ethylhexyl stearate and ethylhexyl hydroxyetarate. The softening agent may include polyethylene waxes such as a polyethylene wax, glyceryl monostearate, sorbitan tristearate, an olefinic thermoplastic elastomer or an amide having the chemical structure CH₃(CH₂)₇CH═CH(CH₂)ₓCONH₂ where x is selected from 5-15. The softening agent may be erucamide CH₃(CH₂)₇CH═CH(CH₂)₁₁CONH₂ which may also be referred to as cis-13-docosenoamide, An example of a commercially available softening agent is erucamide sold under the trademark ARMOSLIP® by Akzo Nobel having an office in Chicago, Ill. Other suggested amide additives include oleylamide CH₃(CH₂)₇CH═CH(CH₂)₈CONH₂ and oleamide N-9-octadecenyl-hexadecanamide) is CH₃(CH₂)₇CH═CH(CH₂)₇CONH₂.

Another group of softening agents includes various silicones and silicone-based compounds that include straight chain, branched and or functionalized silicones. Silicones are polymers that include silicon together with carbon, hydrogen, oxygen, and sometimes other chemical elements. Some common forms include silicone oil, silicone grease, silicone rubber, and silicone resin. More precisely called polymerized siloxanes or polysiloxanes, silicones are mixed inorganic-organic polymers. These materials consist of an inorganic silicon-oxygen backbone ( . . . —Si—O—Si—O—Si—O— . . . ) with organic side groups attached to the silicon atoms, which are four-coordinate. In some cases, organic side groups can be used to link two or more of these —Si—O— backbones together. Sometimes, the silicones contain various functional groups including halogens or amino groups. By varying the —Si—O— chain lengths, side groups, and crosslinking, silicones can be synthesized with a wide variety of properties and compositions. They can vary in consistency from liquid to gel to rubber to hard plastic. The most common siloxane is linear polydimethylsiloxane (PDMS), a silicone oil. The second largest group of silicone materials is based on silicone resins, which are formed by branched and cage-like oligosiloxanes.

Suitably, when the softening agent is being primarily used to improve the transparency and thus the visibility and clarity of the graphics on the inner surface 26 of the film layer 22, the softening agents should be substances which have a viscosity of from about 10 centipoise to about 10,000 centipoise at ambient temperature so they can be applied as fluids to the substrates if so desired.

Suitable softening agents for transparency may include cetyl 2-ethylhexanone, ethylhexyl stearate, and ethylhexyl hydroxyetarate. The softening agent may include polyethylene waxes such as a polyethylene wax and glyceryl monostearate Another group of softening agents includes various silicone fluids that include straight chain, branched and or functionalized silicones. Some examples of such silicones include: dimethicone (DOW CORNING® 365), amino functional silloxane (DOW CORNING® 28818), dimethyl, methylglycol siloxane (Dow Corning® 2-5563), methylhydrogen siloxane (Dow Corning® 75 SF). Other materials that will work well in this regard are lightweight oils such as mineral oils. While such oils can be applied to both the fibrous web 14 and the film layer 22, it may be desirable to only use such materials directly on the inner surface 26 of the film layer 22 to minimize contact with clothing and other materials that may come in contact with the end product employing the laminate. Consequently, as mentioned previously, it may be desirable to employ two different softening agents, one for the exterior of the web 14 and a second for the film 22.

Referring again to FIGS. 3a through 3c, for improving the softness of the outer visible surface 18 of the web 14, the softening agent is topically applied to the nonwoven at an add-on level that suitably improves the softness of the printed nonwoven material. In this regard, the basis weight add-on of the softening agent is from about 0.1 to about 6 grams per square meter, optionally from about 0.1 to about 4 grams per square meter. The higher end of the add-on ranges usually works better when the softening agent is a solid at room temperature.

When adding the softening agent with respect to the embodiments of FIGS. 4 through 6 for the purpose of improving the visibility of the graphics by improving the transparency of the film layer 22, typically, it has been found that the add-on levels between about 0.5 gsm and about 3.0 gsm are suitable.

The particular method of applying the softening agent to the printed surface of the nonwoven web or to the film layer can be any suitable treatment application method, including flexographic, gravure, offset, and ink-jet printing, slot die coating, melt spraying, solvent spray coating, thermal spray coating, meltblowing, dip and squeeze and so forth. The application method can be used to apply the softening agent in any design, figure, or other image on the printed surface of the nonwoven web or the film layer. For example, the softening agent may be placed in controlled or patterned areas on the nonwoven web, applied to the nonwoven web as a monolithic film, or applied to the nonwoven web as particulates. The same is also true with the film layer. The softening agent can be applied neat or from a solvent system. Further the softening agent may be applied at ambient temperature or may be heated for application to the nonwoven or film sheet material.

Application of the softening agent to the printed surface of the nonwoven web suitably improves the softness of the printed and treated nonwoven web as measured by the static coefficient of friction test. For example, the static coefficient of friction, measured as described below, may be reduced to less than about 0.5. In other embodiments, the static coefficient of friction may be reduced to about 0.48 or less, desirably about 0.46 or less, more desirably to about 0.44 or less, or even more desirably to about 0.4 or less. In another aspect, application of the softening agent to the printed surface of the nonwoven web may reduce the static coefficient of friction by about 20 percent or more as compared to the static coefficient of friction measured of a printed nonwoven web not treated with the softening agent. In other embodiments, the application of the softening agent to the printed surface of the nonwoven web may reduce the static coefficient of friction by about 25 percent or more, desirably by about 30% or more, more desirably by about 35% or more, or even more desirably by about 40% or more as compared to the static coefficient of friction measured of a printed nonwoven web not treated with the softening agent.

Application of the softening agent to graphics on the printed surface of the sheet material or nonwoven web desirably will not impact the crockfast resistance of the printed graphics. In some embodiments, the oil crockfastness of the printed surface treated with softening agent remains greater than about 3.0, desirably greater than about 4.0.

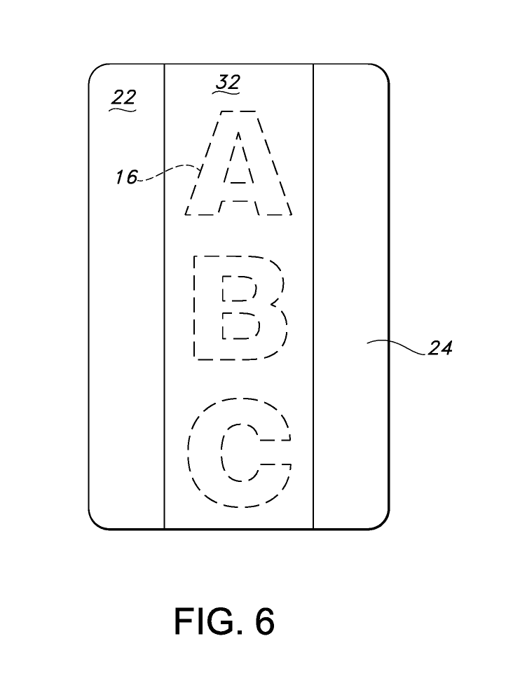

An alternate embodiment of the present invention is shown in FIGS. 4 through 6. FIG. 4 is a cross-sectional side view in an exploded form showing the location of the graphics on the inner surface 26 of the film layer 22 as well as the various surfaces of each of the layers similar to that shown in FIG. 2 for the other embodiment. FIG. 5 shows the layers in FIG. 4 in an assembled state and with an additional layer 32 of the softening agent located on the inner surface 26 of the film layer 22. FIG. 6 is a top plan view of the film layer 22 showing the graphics 16 being visible through the film layer 22 and a localized application of the softening agent 32 down the central portion of the film layer 22 on the outer surface 24 so as to be in vertical juxtaposition with and only cover the area with the printed graphics 16 located on the inner surface 26.

In this embodiment, the problem of ink rub-off and crockfastness is greatly reduced by printing the graphics 16 on the inside of the backsheet 12. More specifically, by printing the graphics 16 on the inner surface 26 of the film 22, the ink is removed from the abrasion that the ink/graphics 16 would otherwise see when printed on the visible outer surface 18 of the web 14. However, printing on this inner surface 26 poses an additional problem in that the graphics 16 become more difficult to see by the end-user or caregiver, especially when the product is a personal care absorbent article such as the training pant 10 shown in FIG. 1 of the drawings.

Printing the graphics on the inner surface 26 of the film layer 22 will typically mean in the context of personal care absorbent articles that the ink will be subject to some type of fluid such as blood, urine, menses or feces. As a result, the nature of the ink formulation must be such that it does not readily dilute or wash off as this will in turn potentially compromise the clarity and visibility of the graphics to the end user unless dilution is intended as when some or all of the ink/graphics are part of a wetness indicator.

When the graphics 16 are located in this position, in order for them to be visible, they must be sufficiently clear so as to be seen through the thickness of the film layer 22 and the nonwoven layer 14. It is not uncommon for both the fibers of the nonwoven web 14 and the film layer 22 to include pigments and/or fillers. In the context of outer covers for absorbent articles, it is desirable for the film layer to be liquid impermeable but breathable to gases and water vapor so as to allow the overall product to have some level of breathability. It is believed by increasing the breathability of the film 22 and thus the backsheet 12, the overall product 10 will in turn be more breathable. This is particularly advantageous in relation to skin wellness and the prevention of diaper rash and other skin disorders. To provide breathability, fillers are often used in the film. The particles are mixed with the film polymer and the film is subsequently stretched to pull portions of the polymer away from the filler particles thereby creating pores within the film to make the film more breathable. Typically such films and other versions of porous films will have pores ranging in size from about 0.1 to about 4.0 microns. The use of such fillers, however, can also increase the opacity of the film thereby impeding the visibility of the graphics printed on the interior surface of the film. In addition to the possible stretching of the film to impart breathability, it is also common for the film layer to be unidirectionally or biaxially stretched to impart further properties to the film such as strength or thinning to reduce cost. Such stretching can sometimes cause partial plastic deformation and stretch whitening of the film yet further reducing transparency and thus, visibility and clarity of the graphics.

During the course of improving the softness of the backsheet 12 through the application of a softening agent 32 to the nonwoven web 14, it was determined that if sufficient quantities of the softening agent 32 were applied to the backsheet 12, some portion of the softening agent 32 migrated to the outer surface 24 of the film 22 and, as a result, the transparency of the film was improved, thereby improving the visibility of the graphics 16 printed on the inner surface 26 of the film 22. While not wishing to be held to a particular theory, it is believed that the softening agent 32 present on the outer surface 24 of the film 22 filled all or a portion of the micro voids in the film 22, thereby improving the transparency or, stated conversely, reducing the opacity of the film layer 22 by changing and improving the refractive index of the film.

As with the embodiments shown in FIGS. 1 through 3c, for the embodiment shown in FIGS. 4 through 6 the same components may be used for the various layers of the overall product 10 including the liquid permeable topsheet 30, the absorbent core 28, the liquid impervious film 22 and the fibrous nonwoven web 14 as well as the inks and graphics 16 and the softening agents 32. The primary difference is that the graphics 16 are printed on the inner surface 26 of the film layer 22 as opposed to the outer visible surface 18 of the nonwoven layer 14. In addition, the same methods of forming the various components and applying one component to another can be utilized in the embodiment shown in FIGS. 4 through 6.

The softening agent 32 can be applied at several locations to achieve the increased visibility of the graphics. For example, in one embodiment, the softening agent can be applied to the outer surface 24 of the film layer 22 prior to the attachment of the nonwoven layer 14 to the film layer 22. In this version, a softening agent should be chosen which does not impede the method by which the nonwoven layer 14 is attached to the film layer 22. Thus, it is possible that one softening agent may be chosen to be added to the film layer 22 and another softening agent may be chosen to be added to the nonwoven layer 14 thereby forming a two-step process for the application of the softening agent 32. Furthermore, one softening agent may be specifically selected for the film layer 22 so as to maximize visibility while a second softening agent is selected for the nonwoven layer 14 to maximize softness.

In another embodiment, the film and nonwoven layers of the laminate or backsheet 12 can be formed in the same way as with the embodiments of FIGS. 3a through 3c with the only variant being the application of the ink/graphics 16 to the inner surface 26 of the film 22. The softening agent 32 is then applied in the same manner (to the outer surface 18 of web 14) but in a sufficient quantity that at least a portion of the softening agent migrates to and at least partially coats the outer surface 24 of the film layer 22. Alternatively, the softening agent 32 can be added using a conventional dip and squeeze method followed by drying in which case all of the fibers as well as the inner surface 20 and the visible outer surface 18 of the web layer 14 and the inner surface 26 and the outer surface 24 of the film layer 22 will be covered and treated with the softening agent 32.

As will be appreciated, some of the softening agents exhibit varying viscosities as compared to one another in the state in which they are being applied to the surface of the film and/or nonwoven layers. Some need to be softened with heat to make them suitable for application while others require some type of solvent so as to be able to be applied by the equipment being used. For example, if the softening agent is to be applied to the outer surface 18 of the nonwoven web 14, it should be sufficiently low in viscosity so as to be able to penetrate through the fibrous layer and migrate down onto the surface of the film for purposes of improving transparency.

Add-on levels of the softening agent can vary depending on the end use and the balance needed between the breathability of the film and the visibility of the graphics. When transparency is the primary attribute being imparted to the laminate 12 or if the outer surface 24 of the film layer 22 is being treated with a select softening agent which is different than the softening agent being applied to the nonwoven layer 14, generally, the softening agent will be present on all or a portion of the film-web laminate in an amount of from about 0.5 to about 3.0 grams per square meter based upon the per unit area of the laminate and not the individual layers or a combination of the surface area of the individual layers. Thus, for example, an add-on of 3.0 grams per square meter would be a total amount of 3.0 grams on a one meter square sample of the laminate irrespective of where on the laminate the softening agent was located. More desirably, the softening agent will be present in an amount of from about 1.0 to about 3.0 grams per square meter. As will be noted from the samples and resultant data below, the add-on value can also be dependent upon the color or colors of the inks and graphics being used. For example, black may require a higher add-on of softening agent. Thus, if there are zones of color printing, it may be possible to use less softening agent on some areas of the laminate as opposed to others to achieve the same level of improvement in optical density than in other areas of the laminate with different colored inks and printing. Therefore, if the ODR with any of the colors meets the prescribed value, for purposes of claim construction, the overall sample is considered to have met the prescribed value.

Another way to reduce the amount of softening agent used on the laminate and more specifically the film layer 22 is to zone coat or treat only the area which contains the graphics 16. An example of this is shown in FIG. 6. In this embodiment, the nonwoven layer 14 is not shown as it has been removed. The graphics 16 are shown being printed on the underside of the film layer 22 and they are located in this instance in the central portion of the film layer 22. As a result, the softening agent need only be applied over this area of the film layer 22 and the outboard areas need not be treated thereby reducing the cost of construction. Depending on the equipment being used, the localized treatment of the film layer 22 may be done directly on the outer surface 24 or it may be done on the same area of the fibrous nonwoven web (not shown) such that the application of the softening agent 32 is in vertical juxtaposition and general registry with the graphics 16.

While the softening agent can improve the visibility of the graphics on films in general, it is particularly advantageous in the context of films that have some degree of breathability. The breathability of a film and a film-web laminate such as are disclosed herein can be determined in accordance with ASTM test method D6701-01.

It has been found, however, that while the softening agents can improve the visibility of the graphics printed on the underside of the film, this improvement in visibility as measured by a reduction in opacity or an increase in optical density for the film and the overall laminate must be balanced with the breathability of the film and resultant film-web laminate. Generally, when employing breathable films as part of the film-web laminate forming the article as defined herein, it is desirable that the film or laminate have a breathability as stated by a water vapor transmission rate of between about 1,000 and about 20,000 grams per square meter per 24 hours (g/m²/24 hrs). It is desirable to have the film and laminate be as breathable as possible in the context of the usage of this material in such items as personal care absorbent articles. As a result, it is desirable that the original breathability of the film and film-web laminate be reduced by no more than about 10% to about 90% when treated with a softening agent as compared to the same material before treatment, more desirably the breathability should be reduced by no more than about 10% to about 50% when possible. If high treatments of the softening agent are required to obtain the desired degree of visibility and clarity, it therefore may be necessary to initially start with a film with a higher degree of breathability than needed so that after treatment, the balance between post-treatment breathability and visibility/clarity are obtained.

The visibility of the laminate may be measured by either or both of the opacity and the optical density of the laminate. Thus, in balancing visibility (when measured by opacity) with breathability, it is desirable that the opacity be reduced (which is an improvement relative to visibility) by about 3% to about 10% when treated with a softening agent as compared to the same material before treatment, more desirably the opacity should be reduced by about 3% to about 20% and most desirably between about 20% and about 50% as compared to a sample not treated with a softening agent. As a result, it is desirable that the laminate have an opacity of between about 40 and about 70, desirably between about 40 and about 60 and more desirably between about 40 and about 55.

With respect to optical density, it is desirable that the laminate have an optical density ratio, (ODR), of between about 30 and about 80, desirably between about 35 and about 80 and more between about 45 and about 80.

### Test Methods

Crockfastness: A crock test method was used to measure whether the combinations of treated nonwovens and inks had sufficient abrasion resistance. The crock test method was based upon American Association of Textile Chemists and Colorists (AATCC) Test Method 116-1983, which is incorporated herein in its entirety with a few modifications, as disclosed in international publication no WO 2004 061200A1.

The AATCC Test Method uses a device called a Rotary Vertical Crockmeter to rub a piece of test fabric against the sample specimen. This modified crock test method used a device called at Sutherland Rub Ink Tester (Sutherland 2000 Rubtester supplied by Danilee Company of San Antonio, Tex.) as an alternative to the crockmeter. The Sutherland Rub Tester is used in the printing industry to evaluate the resistance of inks and coatings on printed substrates. It has a broader test area than the crockmeter. The test head is 2-inches×4-inches for an eight square inch test area. The test head is moved laterally over the test specimen in a shallow arc pattern. Various weights are available to alter the pressure on the test surface and the number of test “strokes” is variable. This test method used a 4.0 pound weight and 50 rub strokes at a frequency of 42 cycles per minute. The test specimen can be abraded against any material that can be readily attached to the opposing surface of the tester.

Under the AATCC method, any transfer of colorant is qualitatively rated from one to five against a standard scale. A five is equivalent to the absence of transfer and a one is equivalent to an extreme amount of colorant transfer. The primary difference between the test method used in the following examples and the AATCC method was a quantitative method of assigning a colorfastness value. The latter was achieved by using a Spectrodensiometer to assign a measurement of total colorant transfer. This measured value is known as “Delta E”. An equation was then developed to convert the Delta E value to into a one to five value equivalent to the AATCC colorfastness scale.

According to the test procedure, test specimens were analyzed for the CIELAB color difference which is expressed as E. The E was then converted to a number between 1 and 5 using the following equation: C. R.=A exp. (−B) where A=5.063244 and B=0.059532 (ΔE) if E is less than 12, or A=4.0561216 and B=0.041218(E) if E is greater than 12. This number C. R. is the crockfastness rating. A rating of 1 corresponds to a low or bad result, while a rating of 5 is the highest possible test result, and this value would indicate that essentially no color was rubbed off the sample material.

With the Spectrodensitometer, greater objectivity in evaluating the results was possible due to less operator dependence, and it was also possible to achieve higher efficiency and consistency for on-line quality assurance. The X-Rite 938 Spectrodensitometer is manufactured by X-Rite, Inc., of Grandville, Mich.

### Equipment and Materials Used

1. Sutherland 2000 Rub Tester (Danilee Co., San Antonio, Tex.). Sharp edges on the vertical rod were filed to reduce abrasion of nonwoven materials.
2. Crockmeter cloth, standard 2-inch by 6-inch (approximately 50 millimeter by 152 millimeter) test squares.
3. Paper Cutter, standard 12-inch by 12 inch (305 mm×305 mm) minimum cutting area, obtained from Testing Machines, Inc., Amityville, N.Y.
4. Room with standard conditions atmosphere: temperature=23±1° C. (73.4±1.8° F.) and relative humidity=50±2 percent. Testing outside the specified limits for temperature and humidity may not yield valid results.
5. X-Rite Spectrodensitometer 938 manufactured by X-Rite, Inc., of Grandville, Mich.

### Specimen Preparation

The test specimens were a spunbond polypropylene web and film laminate having a basis weight of about 1 ounce per square yard. The test specimens were cut exactly 2.5 inches wide by 7.0 inches long, unless otherwise noted, with the test area centered on the square.

### Testing Procedure

1. Cut samples approximately 2.5 inches wide by 7.0 inches long in the machine direction of the substrate unless otherwise noted in the special instructions.
2. Label a white 2-inch×6-inch cotton sheet with the individual sample information.
3. Place the white cotton sheet lengthwise parallel to the direction of the rub. Adhere sample to the base of the machine so that the printed surface faces up and the area to be tested is centered.
4. Weigh one piece of the crockmeter cloth. Thoroughly wet out the crockmeter cloth with baby oil, bringing the wet pickup to 65%+/−5% [% wet pickup=((weight_{wet}−weight_{dry})/weight_{dry})×100]. (When measuring the dry crockfastness, this step can be omitted. When measuring the wet crockfastness, water is substituted for baby oil.)
5. Adhere white crockmeter cloth to 4.0 pound weight by placing the sample to be tested (matching long side to long side) on the weight and taping the excess with 610 tape. Be sure that the sample is taught and the printed side of material is to be facing out when taped on to the weight.
6. Place the weight (4.0 pounds) and white cloth sample on the rub tester arm.
7. Set the rub tester for 50 rub strokes at 42 cycles per minute.
8. Start the rub tester and wait for the tester to stop.
9. Allow the tested sample to dry.
10. When the rub test for the sample is completed, staple the sample to the white cotton cloth with to a sheet of cardboard beneath the sample behind the cloth.
11. Once the rub testing for a batch of samples is completed Spectrodensiometer reading may begin. However, samples that were tested with water or oil must be allowed to dry in an open area of 24 hours before Spectrodensiometer testing.
12. Be sure that the illuminate is set to C²
13. Calibrate the Spectrodensiometer on the white spot using the tile provided.
14. Be sure that the data mode for the printout is set to Difference mode and that it is D50/10 and Lab.
15. A white standard must be read in each new day of Spectrodensiometer reading or more frequently if noted in special instructions. This is done by piling 7 cotton cloths on top of each other and setting the reference with this.
16. Read each sample, reading the area that appears to have the most amount of ink transfer, beginning with the white standard if necessary then proceeding through the batch.
17. Number the sample during the reading consecutively from 1 to the end with number 1 being the white standard if necessary. These numbers should match the printout.
18. After reading all of the samples with the Spectrodensiometer, print out the report and label the report with sample information. (i.e. White standard and Sample identity.)

### Optional Wet Sample Testing:

1. Weigh the Crockmeter cloth standard. Record the weight.
2. Thoroughly wet out the material with the appropriate solution.
3. Bring the wet pick-up to 65+/−. 5 percent (This is done by wringing or blotting the excess solution from the material, weighing the material and calculating the percent pick-up. Calculate: wet weight minus dry weight divided by dry weight times 100=percent pick-up). To prevent evaporation, prepare one wet cloth at a time for testing.
4. Proceed with Steps 4 through 18.

### Evaluation

The next step is the second modification to the AATCC test procedure, as earlier described above. The second modification is that the amount of color transferred to the test specimen was measured using an X-Rite Spectrodensitometer, instead of the AATCC Chromatic Transference Scale or a grade scale measuring device. As earlier described, E is then obtained and converted to a crockfastness rating between 1 and 5 using the equation set forth above.

Each specific sample was tested multiple times to obtain an average reading. The average was determined by individually calculating the crockfastness rating for each of the test specimens, summing the crockfastness ratings, and then dividing by the number of samples to get the average crockfastness rating.

Coefficient of Friction: Coefficient of Friction testing was done in accordance with ASTM D 1894-78 using a high gloss smooth vinyl tile sliding surface.

Water Vapor Transmission Rate: Breathability as measured by water vapor transmission rate is performed in accordance with ASTM D6701-01. Testing is done according to the written test procedure with the following notations. Testing is done at 23 degrees Centigrade using a MOCON® Permatran-W® Model 100K testing apparatus from Mocon, Inc. of Minneapolis, Minn. As permeability and permeance are not being calculated, thickness is not measured in accordance with section 11.2 of the procedure. The samples are not conditioned relative to section 11.6.1. When the samples are laminates, they are loaded into the apparatus with the film side down or to the gas side of the equipment in accordance with the instructions in section 11.3 of the procedure. A total of four cycles of the cells are run relative to the provisions of section 11.6.2 to obtain the water vapor transmission rate which is reported in grams per square meter per 24 hours (day).

Opacity and Optical Density: Both the opacity and optical density of materials are measured using a Datacolor Spectrflash® SF-600 Plus-CT spectrometer from Datacolor of Lawrenceville, N.J. equipped with ChromaCalc® version 3 software. This equipment following its instruction manual is equipped to generate both opacity and optical density readings for materials such as are disclosed herein. Initial set-up of the apparatus should include setting measurements being taken to include both specular light and UV light. Under the setting for “Method of Strength Adjustment” the software should be set to “Maximum Absorption Peak”. The “Illuminant/Observer Conditions” are set to “D65 10 degrees”. Results as to opacity are reported herein as “batch opacity” which is generated through the use of the “BatCR/Opacity/Haze” data field in the apparatus software. Results as to optical density are reported herein as “batch strength” which is generated through the use of the “strength difference” data field in the apparatus software.

Calibration should be done as per the instruction manual on the same day before any measurements are taken.

For both opacity and optical density measurements, four sample measurements are taken from either four separate samples of material or four separate areas of the same sample material depending on the size of the available sample area. Each of the four sample measurements should be taken of the same type and color of area. For opacity, which is a measure of light reflectance of the background object, measurements should be taken in a plain area of the material that does not contain any ink or graphics. For optical density, each of the four measurements should be taken from an area with the same color and where the selected color area is sufficiently large so as to completely cover the viewing aperture of the testing apparatus. In this regard, it should be noted the apparatus has interchangeable viewing apertures of different sizes which can be interchanged to meet this requirement but all sample measurements for a particular color should be taken using the same aperture size. Available aperture sizes include, but are not limited to, “USAV 6.6 mm”, SAV 9.0 mm″ and “LAV 30 mm”. Note that each time an aperture size is changed, the apparatus must be recalibrated.

Opacity

Measurements and data for opacity can be taken with either the nonwoven side or the film side of the film-nonwoven laminate adjacent the optical port of the testing apparatus as the measurement being taken is the overall opacity of the laminate. For the purposes of the batch opacity data reported herein (Table 7), the fiber side of the film-web laminate was placed adjacent the optical port.

For each sample, four sets of measurements are taken. Four measurements are taken using the white calibration tile inserted into the spring-loaded sample arm holder and four measurements are taken using the black trap inserted into the spring-loaded sample arm holder. Again, the aperture size should be selected so that the viewing area does not contain any inks or other graphics. The software then generates a single averaged value (the “batch opacity”) which is reported as a number based on a completely opaque material being 100 and a completely translucent material being 0. Thus, as the opacity decreases, the material is less opaque and thus more translucent and better able to permit viewing of the printed graphics.

Optical Density

For optical density, neither the white tile nor the black trap is used with the spring-loaded sample arm holder. As a result, only four measurements are taken for each color using the above described procedures for sample placement and aperture size selection. To generate the batch standard for the optical density readings in Table 6, first a control sample is measured for each color. For Table 6 the standard for each color was an untreated but printed sample of the above-described film-web laminate with the fiber side of the film-web laminate positioned against the optical port. This was a sample that had not gone through the dip and squeeze process. Once the batch standard has been recorded for each color, the four batch sample readings were taken for each color at each of the six softening agent add-on levels (0 gsm, 0.62 gsm, 0.87 gsm, 1.05 gsm, 1.48 gsm and 2.0 gsm). For each color, the software then generated a single averaged batch strength value for these four measures which is reported as a dimensionless number in Table 6. A higher number for optical density is an indication that the density of the ink or graphic is higher and thus the ink or graphic is more visible and more vibrant than a lower value.

Optical Density Ratio

For the optical density values reported in Table 6, each of the batch strengths for each of the colors was generated at each of the add-on levels using the same, single standard for each color. In Table 8, each batch strength for each color at each add-on level was based on a standard for each color at each add-on level. For example, for the batch strength value of 23.91 for the color orange in Table 8 at an add-on level of 0.25 gsm of polysiloxane, first a standard was obtained by measuring the color orange with the film side of the 0.25 gsm film-web sample placed adjacent the optical port. Once this had been done, four batch samples were generated by taking four measurements of the color orange from the fiber side of the 0.25 gsm film-web laminate sample and the results of these measurements (the batch strength of 23.91) was obtained and recorded for this add-on level in Table 8. This value is termed the “optical density ratio” or “ODR” to differentiate this measurement technique from that used with respect to Table 6. The ODR is a means of comparing the optical density of the color from the printed side of a sample with the optical density of the color when viewed from the fiber side of the same sample.

Optical Density Ratio Improvement

The improvement in the optical density can be determined by comparing the optical density ratios of the treated and untreated samples. The improvement of the optical density ratio can be determined as follows: optical density ratio improvement (ODRI)=(ODR of printed, treated sample)−(ODR of printed, untreated sample). If an untreated sample is not available, and if the majority (in excess of 80 weight percent) of the softening treatment can be removed from the sample using solvent washing techniques, the sample will be considered as an untreated sample for purposes of calculating the ODRI.

Softening Agent Add-On

The amount of added softening agent to the laminate may be determined by various analytical tools available to one skilled in the art. Non-limiting examples of testing methods include direct spectroscopic measurements of treated samples using different spectroscopic methods such as Raman Microspectroscopy. Alternatively, the softening agent level can be measured after extracting the softening agent from the sample with appropriate solvents suitable for the softening agent. Once the softening agent has been extracted, further analysis can be conducted using appropriate instrumentation such as, for example, chromatographic separation followed by detection, or elemental analysis. These same extraction techniques can be used to create untreated samples for optical density and opacity measurements and analysis. For more information on extraction and analysis of silicones see “Inorganic Polymers” edited by Roger De Jaeger (Lab. de Spechtrochimie Infrarouge et Raman, Univ. des Sciences and Tecn. de Lille, France); Mario Gleria (Inst. di Scienze e Tecn. Molecolari, Univ. di Padoa, Italy), Nova Publishers (2008) and in particular, Chapter 2 entitled “Silicones in Industrial Applications” and the article published therein entitled “Characterization of Silicones” which is incorporated herein in its entirety.

For purposes of calculating opacity, optical density, optical density ratio and optical density ratio improvement, if any portion of the sample under review meets a prescribed value, for purposes of claim construction, the overall sample is considered to have met the prescribed value.

### Examples

Relative to the embodiments shown in FIGS. 3a through 3c, printed adhesively-bonded spunbond/film laminates (aSFL) and thermally-bonded breathable stretch-thinned spunbond/film laminates (bSTL), substrates, were coated with erucamide by spraying molten erucamide on to the substrates with heated air. The erucamide used was from Akzo Nobel with the brand name Armoslip-E. Different add-on levels were obtained by running the substrates at different speeds. Add-on levels were determined by determining differences in weight measurements of the coated and uncoated substrates. For aSFL printed with acrylic-based inks available from Polytex Environmental Inks containing crosslinker PENTAERYTHRITOL-TRIS-(B-(AZIRIDINYL)PROPIONATE) (XAMA 7, produced by Noveon), add-on levels from 1.3 gsm to 5.8 gsm were obtained. On the bSTL with graphics created with Arrowflex inks available from Flint, add-on levels from 2.6 gsm to 10 gsm were obtained. The two substrates had different widths, resulting in different add-on levels.

Crockfastness (CR) was measured on the substrates, as described above, to determine the impact of erucamide on rub resistance. The results are shown in Table 1.

TABLE 1
Crockfastness

| | Add-on | Crockfastness data | | | | | |
|---|---|---|---|---|---|---|---|
| Sample/ | level, | CR | Stdev. | CR | Stdev. | | Stdev. |
| linespeed | g/m² | Dry | Dry | Wet | Wet | CR Oil | Oil |

Substrate: aSFL printed as described above

| | | | | | | | |
|---|---|---|---|---|---|---|---|
| Control | 0.0 | 4.78 | 0.04 | 4.77 | 0 | 4.47 | 0.11 |
| 120 ft/min | 1.3 | 4.8 | 0.01 | 4.83 | 0.02 | 4.58 | 0.02 |
| 90 ft/min | 1.6 | 4.78 | 0.06 | 4.84 | 0.04 | 4.53 | 0.09 |
| 60 ft/mi | 2.7 | 4.78 | 0.02 | 4.87 | 0.02 | 4.5 | 0.09 |
| 45 ft/min | 3.7 | 4.76 | 0.09 | 4.87 | 0.03 | 4.42 | 0.03 |
| 30 ft/min | 5.8 | 4.8 | 0.04 | 4.82 | 0.02 | 4.61 | 0.02 |

Substrate: bSTL printed as described above

| | | | | | | | |
|---|---|---|---|---|---|---|---|
| Control | 0 | 4.53 | 0.08 | 4.47 | 0.1 | 2.54 | 0.12 |
| 120 ft/min | 2.6 | 4.44 | 0.29 | 4.77 | 0.1 | 2.81 | 0.27 |
| 90 ft/min | 3.6 | 4.78 | 0.06 | 4.85 | 0.07 | 2.89 | 0.22 |
| 60 ft/mi | 6.0 | 4.66 | 0.15 | 4.76 | 0.15 | 2.68 | 0.19 |
| 45 ft/min | 8.2 | 4.74 | 0.04 | 4.75 | 0.2 | 2.47 | 0.09 |
| 30 ft/min | 10.1 | 4.6 | 0.08 | 4.77 | 0.08 | 2.56 | 0.29 |

The results show that erucamide spray coating has minimal or no negative impact on crockfastness of the printed bSTL or aSFL. There is even a directional improvement in wet rub resistance (crockfastness).

Again referring to the embodiments shown in FIGS. 3a through 3c, erucamide (Armoslip-E) was coated at various add-on levels onto printed aSFL material (printed 12 gsm spunbond polypropylene (SFT-315 polypropylene available from Exxon-Mobil Chemical Company) fibers laminated with 1 gsm Rextac 2215 adhesive (available from Huntsman Polymers of Houston, Tex.) to a breathable 17 gsm LLDPE stretch-thinned film containing 60% CaCO₃ with 1 micron average particle size) by a slot coating process four hours after printing. Crockfastness (CR) was measured on the substrates, as described above, to determine the impact of erucamide on rub resistance. The results are shown in Table 2. Softness was evaluated by measuring Coefficient of Friction as described above. The results are shown in Table 3.

TABLE 2
Crockfastness

| | | | | Wet | CR | Oil |
|---|---|---|---|---|---|---|
| Add-on level, g/m2 | CR Dry | Dry stdev | CR Wet | Stdev | Oil | Stdev |
| Control - 0 gsm | 4.87 | 0.03 | 4.83 | 0.05 | 3.56 | 0.08 |
| 1 gsm | 4.83 | 0.03 | 4.92 | 0.03 | 3.44 | 0.24 |
| 2 gsm | 4.86 | 0.03 | 4.91 | 0.01 | 3.56 | 0.11 |
| 3 gsm | 4.84 | 0.03 | 4.95 | 0.02 | 3.99 | 0.08 |
| 4 gsm | 4.92 | 0.03 | 4.91 | 0.03 | 3.96 | 0.02 |

TABLE 3
Softness

| | Coefficient of Friction (COF) | | | | | |
|---|---|---|---|---|---|---|
| | Peak Load | | | | Dynamic | |
| Add-on | (gf) | | Static COF | | (kinetic) COF | |
| level, g/m2 | average | std dev | average | std dev | average | std dev |
| Control - | 147 | 21 | 0.74 | 0.11 | 0.33 | 0.03 |
| 0 gsm | | | | | | |
| 1 gsm | 100 | 18 | 0.51 | 0.09 | 0.34 | 0.06 |
| 2 gsm | 99 | 12 | 0.50 | 0.06 | 0.30 | 0.03 |
| 3 gsm | 92 | 4 | 0.46 | 0.02 | 0.31 | 0.03 |
| 4 gsm | 88 | 10 | 0.44 | 0.05 | 0.32 | 0.03 |

Again referring to the embodiments shown in FIGS. 3a through 3c, erucamide was coated at various add-on levels onto printed aSFL material as described above by a slot coating process after printing. Crockfastness (CR) was measured on the substrates, as described above, to determine the impact of erucamide on rub resistance. The results are shown in Table 4. Softness was evaluated by measuring Coefficient of Friction (gf) as described above. The results are shown in Table 5.

TABLE 4
Crockfastness

| | Dry | Dry | Wet | Wet | Oil | Oil |
|---|---|---|---|---|---|---|
| Add-on level | CR | Stdev | CR | Stdev | CR | Stdev |
| Control-0 gsm | 4.87 | 0.02 | 4.84 | 0.02 | 4.27 | 0.08 |
| 2 gsm | 4.97 | 0.05 | 4.92 | 0.01 | 4.14 | 0.06 |
| 6 gsm | 4.96 | 0.00 | 4.97 | 0.02 | 4.44 | 0.13 |
| 8 gsm | 4.94 | 0.01 | 4.94 | 0.04 | 4.24 | 0.07 |
| 10 gsm | 4.94 | 0.04 | 4.97 | 0.04 | 4.54 | 0.10 |

TABLE 5
Softness

| | Coefficient of Friction (gf) | | |
|---|---|---|---|
| | Peak | Static | Dynamic |
| Add-on level, g/m² | Load | COF | (kinetic) COF |
| 0 gsm | 86.9 | 0.44 | 0.21 |
| 2 gsm | 80.2 | 0.41 | 0.31 |
| 4 gsm | 79.8 | 0.40 | 0.30 |
| 6 gsm | 72.9 | 0.37 | 0.25 |
| 8 gsm | 73.9 | 0.37 | 0.22 |
| 10 gsm | 70.1 | 0.35 | 0.21 |

As can be seen in the Tables, crockfastness generally increased (improved) and Static Coefficient of Friction generally decreased (improved) as the amount of softening agent (erucamide) that was applied over the printed graphic was increased.

Relative to the embodiments shown and described with respect to FIGS. 4 through 6, an adhesively-bonded spunbond/film (aSFL) laminate material was formed from a 12 gsm fibrous spunbond web made from “3155” polypropylene available from the Exxon-Mobil Chemical Company. The resultant web was laminated using 1 gsm Rextac 2215 adhesive (available from Huntsman Polymers of Houston, Tex.) to a breathable 17 gsm LLDPE stretch-thinned film containing 60% CaCO₃ with a 1 micron average particle size. The spunbond was pre-bonded before lamination to the film layer. The inks used to print the laminate were acrylic-based inks available from Polytex Environmental Inks containing crosslinker PENTAERYTHRITOL-TRIS-(B-(AZIRIDINYL)PROPIONATE) (XAMA 7, produced by Noveon). The inks were applied using flexographic printing equipment. Colors used were blue, green orange and black. Separate figures using single colors were printed in a pattern of the type shown in FIG. 1 though different characters were used.

The samples were printed on the interior side of the film corresponding to inner surface 26 in the drawings. The softening agent was added using a standard dip and squeeze process followed by drying. The solution to which the softening agent was added was water with 15% by weight isopropyl alcohol. The sample with 0 gsm of softening agent was run through the dip and squeeze process prior to the addition of the softening agent. The other samples were treated with increasing add-ons of a polysiloxane emulsion (Dow Corning® 75SF emulsion from Dow Corning Corporation of Midland, Mich.). (According to the MSDS sheet for this emulsion it contains from 40.0 to 60.0 weight percent methylhydrogen siloxane, 30.0 to 50.0 weight percent water, 5.0 to 10.0 weight percent hydroxyl-terminated dimethyl siloxane with some variantions containing from 1.0 to 5.0 weight percent polyethylene oxide lauryl ether.) The amount of dry weight add-on of the polysiloxane was measured by weighing an approximately 100 millimeter by 100 millimeter square sample before and after the dip and squeeze treatment and drying. The amount of polysiloxane in grams was then reported on a gram per square meter (gsm) basis. As a result of the dip and squeeze application method, all surfaces of the film and nonwoven web were covered. It is possible, however, to use other application techniques, such as spraying, to reduce the amount and location of the application.

The optical density of the samples was measured for each of the four colored objects and the optical density values (an average of four readings) are reported in Table 6. Density measurements were taken with the nonwoven side of the samples facing the optical port of the test instrument. Samples 1 through 6 were film-printed as noted above. Sample 1 was a control with no polysiloxane treatment. The untreated film printed sample, Sample 1, in each case for each color had the lowest optical density value. With increasing softening agent add-on, the optical density of the samples began to increase.

TABLE 6
Optical Density

| | | Polysiloxane | Ink | Ink | Ink | Ink |
|---|---|---|---|---|---|---|
| | Ink | add-on | Color | Color | Color | Color |
| Sample | Location | (gsm) | Blue | Green | Orange | Black |
| 1 | Printed on | 0 | 105.13 | 117.41 | 118.42 | 115.28 |
| | film | | | | | |
| 2 | Printed on | 0.62 | 169.56 | 144.79 | 145.09 | 154.32 |
| | film | | | | | |
| 3 | Printed on | 0.87 | 175.11 | 175.71 | 161.65 | 188.27 |
| | film | | | | | |
| 4 | Printed on | 1.05 | 190.8 | 215.21 | 199.12 | 236.19 |
| | film | | | | | |
| 5 | Printed on | 1.48 | 190.22 | 223.39 | 230.43 | 208.87 |
| | film | | | | | |
| 6 | Printed on | 2.00 | 252.25 | 198.23 | 264.13 | 326.23 |
| | film | | | | | |

In addition to optical density measurements, it is also possible to quantify the advantages of the softening agent in the context of the reduction in the opacity of the film-web laminate. Sample preparation for opacity measurements were the same as those outline above for the optical density samples, the only difference being the test method used and the location of the test measurement on the samples. As indicated in the test procedures, optical density is measured in vertical juxtaposition with the ink printing while opacity measurements are taken in an area where no ink is present.

As can be seen from the results in Table 7, as the amount of softening agent add-on was increased, the opacity of the sample went down thereby indicating a higher degree of visibility with respect to the samples.

TABLE 7
Opacity

| | | Polysiloxane | |
|---|---|---|---|
| | Ink | add-on | Batch |
| Sample | Location | (gsm) | Opacity |
| 1 | Printed on | 0 | 75.03 |
| | film | | |
| 2 | Printed on | 0.62 | 69.38 |
| | film | | |
| 3 | Printed on | 0.87 | 68.04 |
| | film | | |
| 4 | Printed on | 1.05 | 60.19 |
| | film | | |
| 5 | Printed on | 1.48 | 62.02 |
| | film | | |
| 6 | Printed on | 2.00 | 55.43 |
| | film | | |

Optical density ratio (ODR) measurements were made on additional film-nonwoven laminates made from the same materials as outlined above with respect to Tables 6 and 7. In each case, the sample was printed on the film side. As can be seen for each of the four colors (orange, cyan, black and green), the optical density ratio increased with increased add-on of the softening agent.

TABLE 8
Optical Density Ratio

| | Polysiloxane | | | | |
|---|---|---|---|---|---|
| | Add-on | Orange | Cyan | Black | Green |
| Sample | (gsm) | ODR | ODR | ODR | ODR |
| 1 | 0 | 13.9 | 30.3 | 4.47 | 19.8 |
| 2 | 0.25 | 23.91 | 36.6 | 5.9 | 19.81 |
| 3 | 0.49 | 22.99 | 36.58 | 5.11 | 24.17 |
| 4 | 0.57 | 30.63 | 40.49 | 6.1 | 25.07 |
| 5 | 1.01 | 34 | 46.99 | 7.79 | 29.36 |
| 6 | 1.43 | 35.77 | 51.78 | 8.21 | 32.02 |

Higher add-ons of softening agent will continue to provide greater transparency and thus greater visibility and clarity. Additional samples were made with the same laminate and softening agent applied via a dip and squeeze method in a range of approximately 3 to 6 gsm and optical density ratios were measured for the above colors (orange, cyan, black and green) at levels of 59.27, 79.75, 42.6 and 72.75 respectively.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged either in whole or in part. Furthermore, those of ordinary skill in the art will appreciate that the foregoing description is by way of example only, and is not intended to limit the invention so further described in the appended claims.

We claim:

1. An article defining a visible surface comprising:
a film having an inner surface and an outer surface;
a fibrous nonwoven web having an outer visible surface and an inner surface with said inner surface of said fibrous nonwoven web attached to said outer surface of said film to form a laminate having a visible surface defined by said visible outer surface of said fibrous nonwoven web;
an ink composition overlaying said inner surface of said film to form a graphic thereon; and,
a softening agent overlaying at least a portion of said outer surface of said film;
said laminate having a water vapor transmission rate of between about 1,000 and about 20,000 grams per square meter per day;
said laminate having at least one of an opacity between about 40 and about 70 or an optical density ratio between about 30 and about 80.

2. The article of claim 1 wherein said ink composition and said softening agent are both applied to said respective inner and outer surfaces of said film in respective patterns such that said ink composition and said softening agent are in vertical juxtaposition with one another.

3. The article of claim 2 wherein said fibrous nonwoven web is treated with a softening agent.

4. The article of claim 1 wherein said softening agent is present in a concentration of about 0.5 to about 3.0 grams per square meter.

5. The article of claim 1 wherein said opacity is between about 40 and about 55.

6. The article of claim 1 wherein said softening agent comprises silicone or a silicone-based compound.

7. The article of claim 1 wherein said softening agent includes one or more of polysiloxane, straight chain silicone, branched chain silicone, functionalized silicone, dimethicone, amino functional siloxane, dimethyl, methylglycol siloxane and methyhydrogen siloxane.

8. The article of claim 7 wherein said softening agent is present in a concentration of about 0.5 to about 3.0 grams per square meter.

9. The article of claim 3 wherein said fibrous nonwoven web and said film are treated with different softening agents.

10. The article of claim 1 wherein said softening agent is present on said outer surface of said film only in areas in general vertical juxtaposition with said ink composition on said bottom surface of said film so as to form areas on said outer surface of said film which are devoid of said softening agent.

11. An absorbent article comprising:
a liquid permeable topsheet;
a backsheet; and
an absorbent core disposed between said topsheet and said backsheet;
said backsheet comprising a film having an inner surface and an outer surface; a fibrous nonwoven web having an outer visible surface and an inner surface with said inner surface of said fibrous nonwoven web attached to said outer surface of said film to form a laminate having a visible surface defined by said visible outer surface of said fibrous nonwoven web; an ink composition overlaying said inner surface of said film to form a graphic thereon; and, a softening agent overlaying at least a portion of said outer surface of said film; said laminate having a water vapor transmission rate of between about 1,000 and about 20,000 grams per square meter per day; said laminate having at least one of an opacity between about 40 and about 70 or an optical density ratio between about 30 and about 80.

12. A method of forming an article defining a visible surface, the method comprising:
providing a film having an inner surface and an outer surface;
providing a fibrous nonwoven web having an outer visible surface and an inner surface;
attaching said inner surface of said fibrous nonwoven web to said outer surface of said film to form a laminate having a visible surface defined by said outer visible surface of said fibrous nonwoven web;
applying an ink composition to said inner surface of said film to form graphics thereon;
applying a softening agent to said outer surface of said film such that said laminate such that said laminate has a water vapor transmission rate of between about 1,000 and about 20,000 grams per square meter per day and such that said laminate has at least one of an opacity between about 40 and about 70 or an optical density ratio of between about 30 and about 80.

13. The method of claim 12 wherein said softening agent is applied to said outer surface of said film in a pattern which is in general registry with said graphics on said inner surface of said film.

14. A method of forming an article defining a visible surface, the method comprising:
providing a film having an inner surface and an outer surface;
providing a fibrous nonwoven web having an outer visible surface and an inner surface;
attaching said inner surface of said fibrous nonwoven web to said outer surface of said film to form a laminate having a visible surface defined by said outer visible surface of said fibrous nonwoven web;
applying an ink composition to said inner surface of said film to form graphics thereon;
applying a softening agent to said fibrous nonwoven web such that at least a portion of said softening agent is present on said outer surface of said film and said such that said laminate has a water vapor transmission rate of between about 1,000 and about 20,000 grams per square meter per day and such that said laminate has at least one of an opacity between about 40 and about 70 or an optical density ratio of between about 30 and about 80.

15. The method of claim 14 wherein said softening agent on said film in present in a pattern which is in general registry with said graphics on said inner surface of said film.
